SEQUENCE LISTING (19 sequences)

SEQ ID NO: 1 - protein, 113 aa, synthetic construct
QVQLKQSGAELMKPGASVKLSCKATGYTFTGKWIEWVKQRPGHGLEWIGEILPGTGSTNYNEKFKGKATFTADSSSNTAYMQLSSLTTEDSAMYYCLRSEDVWGTGTTVTVSS

SEQ ID NO: 2 - protein, 113 aa, synthetic construct
DIVMTQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNIPTFGGGTKLEIKR

SEQ ID NO: 3 - protein, 5 aa, synthetic construct
GKWIE

SEQ ID NO: 4 - protein, 17 aa, synthetic construct
EILPGTGSTNYNEKFKG

SEQ ID NO: 5 - protein, 4 aa, synthetic construct
SEDV

SEQ ID NO: 6 - protein, 17 aa, synthetic construct
KSSQSLLNSRTRKNYLA

SEQ ID NO: 7 - protein, 7 aa, synthetic construct
WASTRES

SEQ ID NO: 8 - protein, 8 aa, synthetic construct
KQSYNIPT

SEQ ID NO: 9 - protein, 113 aa, synthetic construct
QVQLVQSGAEVKKPGASVKVSCKASGYTFTGKWIEWVRQAPGQGLEWIGEILPGTGSTNYAQKFQGRATFTADSSTSTAYMELSSLRSEDTAVYYCLRSEDVWGQGTLVTVSS

SEQ ID NO: 10 - protein, 113 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCKQSYNIPTFGQGTKVEIKR

SEQ ID NO: 11 - protein, 17 aa, synthetic construct
EILPGTGSTNYAQKFQG

SEQ ID NO: 12 - protein, 113 aa, synthetic construct
QVQLVQSGAEVKKPGASVKVSCKASGYTFTGKWIEWVRQAPGQGLEWIGEILPGTGSTNYNEKFKGRATFTADSSTSTAYMELSSLRSEDTAVYYCLRSEDVWGQGTLVTVSS

SEQ ID NO: 13 - protein, 113 aa, synthetic construct
DIVMTQSPDSLAVSLGERATINCKSSQSLLASRTRKNYLAWYQQKPGQPPKLLIYWASTRESGVPDRFSGSGSGTDFTLTISSLQAEDVAVYYCKQSYNIPTFGQGTKVEIKR

SEQ ID NO: 14 - protein, 17 aa, synthetic construct
KSSQSLLASRTRKNYLA

SEQ ID NO: 15 - protein, 219 aa, synthetic construct
DIVMTQSPDSLAVSLGERATINCKSSQSLLASRTRKNYLAWYQQKPGQPPKLLIYWASTRESGVPDRFSGSGSGTDFTLTISSLQAEDVAVYYCKQSYNIPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 440 aa, synthetic construct
QVQLVQSGAEVKKPGASVKVSCKASGYTFTGKWIEWVRQAPGQGLEWIGEILPGTGSTNYNEKFKGRATFTADSSTSTAYMELSSLRSEDTAVYYCLRSEDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 17 - protein, 728 aa, Homo sapiens
MRWLLLYYALCFSLSKASAHTVELNNMFGQIQSPGYPDSYPSDSEVTWNITVPDGFRIKLYFMHFNLESSYLCEYDYVKVETEDQVLATFCGRETTDTEQTPGQEVVLSPGSFMSITFRSDFSNEERFTGFDAHYMAVDVDECKEREDEELSCDHYCHNYIGGYYCSCRFGYILHTDNRTCRVECSDNLFTQRTGVITSPDFPNPYPKSSECLYTIELEEGFMVNLQFEDIFDIEDHPEVPCPYDYIKIKVGPKVLGPFCGEKAPEPISTQSHSVLILFHSDNSGENRGWRLSYRAAGNECPELQPPVHGKIEPSQAKYFFKDQVLVSCDTGYKVLKDNVEMDTFQIECLKDGTWSNKIPTCKIVDCRAPGELEHGLITFSTRNNLTTYKSEIKYSCQEPYYKMLNNNTGIYTCSAQGVWMNKVLGRSLPTCLPECGQPSRSLPSLVKRIIGGRNAEPGLFPWQALIVVEDTSRVPNDKWFGSGALLSASWILTAAHVLRSQRRDTTVIPVSKEHVTVYLGLHDVRDKSGAVNSSAARVVLHPDFNIQNYNHDIALVQLQEPVPLGPHVMPVCLPRLEPEGPAPHMLGLVAGWGISNPNVTVDEIISSGTRTLSDVLQYVKLPVVPHAECKTSYESRSGNYSVTENMFCAGYYEGGKDTCLGDSGGAFVIFDDLSQRWVVQGLVSWGGPEECGSKQVYGVYTKVSNYVDWVWEQMGLPQSVVEPQVER

SEQ ID NO: 18 - protein, 5 aa, synthetic construct
GGGGS

SEQ ID NO: 19 - protein, 14 aa, synthetic construct
GGGGSGGGGSGGGG

(19) United States
(12) Patent Application Publication
(10) Pub. No.: US 2024/0228666 A1
(43) Pub. Date: Jul. 11, 2024

(54) THERAPEUTIC METHODS AND USES FOR ANTIBODIES TO HUMAN MASP-3

(71) Applicant: Omeros Corporation, Seattle, WA (US)

(72) Inventors: William Jason Cummings, Kirkland, WA (US); William Pullman, Plymouth, MN (US); John Steven Whitaker, Seattle, WA (US)

(21) Appl. No.: 18/498,508

(22) Filed: Oct. 31, 2023

Related U.S. Application Data
(60) Provisional application No. 63/381,975, filed on Nov. 2, 2022.
(60) Provisional application No. 63/383,000, filed on Nov. 9, 2022.
(60) Provisional application No. 63/497,503, filed on Apr. 21, 2023.
(60) Provisional application No. 63/507,304, filed on Jun. 9, 2023.

(51) Int. Cl. C07K 16/40; A61P 7/06; A61P 13/10

(52) U.S. Cl. CPC C07K 16/40; A61P 7/06; A61P 13/10; C07K 2317/24

(57) ABSTRACT

Methods of treating paroxysmal nocturnal hemoglobinuria, complement 3 glomerulopathy, or idiopathic immune complex-mediated glomerulonephritis using MASP-3 serine protease inhibitors are provided. In some embodiments, the MASP-3 serine protease inhibitors are anti-MASP-3 antibodies. Also provided are uses of MASP-3 serine protease inhibitors in treatment of paroxysmal nocturnal hemoglobinuria, complement 3 glomerulopathy, or idiopathic immune complex-mediated glomerulonephritis and for manufacture of a medicament for treatment of paroxysmal nocturnal hemoglobinuria, complement 3 glomerulopathy, or idiopathic immune complex-mediated glomerulonephritis.

### FIELD OF THE INVENTION

The present invention relates to uses of antibodies or antigen-binding fragments thereof that specifically bind to human MASP-3.

### STATEMENT REGARDING SEQUENCE LISTING

The sequence listing associated with this application is provided in XML format in lieu of a paper copy and is hereby incorporated by reference into the specification. The name of the XML file containing the sequence listing is: MP_1_0337_Sequence Listing_20231023_ST26.xml. The XML file is 19,552 bytes; was created on Oct. 23, 2023; and is being submitted via the Patent Center with the filing of the specification.

### BACKGROUND

The complement system provides an early acting mechanism to initiate, amplify and orchestrate the immune response to microbial infection and other acute insults (M. K. Liszewski and J. P. Atkinson, 1993, in Fundamental Immunology, Third Edition, edited by W. E. Paul, Raven Press, Ltd., New York) in humans and other vertebrates, and also has a role in immune surveillance against cancer (P. Macor, et al., Front. Immunol., 9:2203, 2018). More than 30 fluid-phase and membrane-bound glycoproteins, cofactors, receptors, and regulatory proteins are involved in the complement system (S. Meyer, et al., mAbs, 6:1133, 2014). Many of them are serine proteases, which form a highly regulated cascade of activation events. The complement system responds rapidly to molecular stress signals through a cascade of sequential proteolytic reactions initiated by the binding of pattern recognition receptors (PRRs) to distinct structures on damaged cells, biomaterial surfaces, or microbial intruders (Reis et al., Nat. Rev. Immunol., 18:5, 2018).

Activation of the complement cascade induces diverse immune effector functions, such as cell lysis, phagocytosis, chemotaxis and immune activation (S. Meyer, et al., 2014). Furthermore, the complement system also acts as a bridge between the innate immune response and the subsequent activation of adaptive immunity. In addition to its anti-infectious properties, the complement system is also involved in the clearance of immune complexes and apoptotic cells, tissue regeneration, mobilization of hematopoietic progenitor cells, and angiogenesis (T. M. Pierpont et al., Front. Oncol., 8:163, 2018).

The complement system can be activated through three distinct pathways: the classical pathway, the alternative pathway, and the lectin pathway. See FIG. 1. Activation of the classical pathway is triggered by a conformational change of the classical pathway initiation complex C1, composed of C1q, a hexamer of trimeric chains, and a heterotetramer of the C1q-associated serine proteases C1r and C1s, as detailed below. The binding of C1q to complexes composed of host antibodies bound to a foreign particle (i.e., an antigen) initiates the activation of C1 complex. Since activation of the classical pathway largely depends on a prior adaptive immune response by the host, the classical pathway is an effector mechanism of the acquired immune system. In contrast, both the lectin and alternative pathways are independent of adaptive immunity and are part of the innate immune system.

The classical pathway (CP) is primarily initiated by antibody-antigen complexes. Antibodies of subclasses IgM and IgG bind to an antigen on the surface of a pathogen or a target cell and recruit the C1 complex, which is composed of the multimolecular recognition subcomponent C1q (composed of six heterotrimers of the C1q A-chain, B-chain, and C-chain) and the C1q-associated serine proteases C1r and C1s. Upon binding of C1q to the Fc-region of either an IgM bound to an antigen or to at least two IgG antibodies bound to their antigens, the serine protease C1r is converted from its zymogen form into its enzymatically active form and subsequently cleaves and activates its substrate C1s. Once activated, C1s cleaves C4 into its fragments C4a and C4b. C4b binds to complement component C2 and this complex, C4bC2, is cleaved by C1s in a second cleavage step to release C2b, forming the complement C3 converting enzyme complex C4bC2a, a so-called C3 convertase, which cleaves the abundant plasma complement component C3 into C3a and C3b.

The lectin pathway is triggered by the binding of pattern recognition molecules, such as mannose-binding lectin (MBL), ficolins or collectin-11 and collectin-10, to pathogen-associated molecular patterns (PAMPs) or apoptotic or distressed host cells. The recognition molecules form a complex with the MBL-associated serine proteases, MASP-1 and MASP-2, and activate them upon binding, which results in the cleavage of C2 and C4 and the formation of the C3 convertase (C4bC2a).

The alternative pathway is initiated by spontaneous hydrolysis of C3 (“tickover”) to C3(H₂O), which binds to factor B (fB). The conversion of the resulting C3(H₂O)fB complexes requires the enzymatic activity of another highly specific serine protease called factor D. The availability of enzymatically active factor D is thought to be a limiting factor for the alternative pathway amplification loop and the availability of factor D requires the action of another enzyme, MASP-3, which is required for conversion of pro-Factor D (proCFD) into its active form, mature factor D (matCFD) (Dobo et al., Sci Rep 6:31877, 2016). Activated mature factor D (matCFD), another serine protease, cleaves the C3(H2O)-bound fB into Ba and Bb. Bb is also a serine protease and participates in the formation of alternative C3 pro-convertases C3(H₂O)Bb and C3bBb, which cleave C3 into C3a and C3b. By this mechanism, the alternative pathway is constitutively active at low levels. The AP amplification loop is formed when freshly generated C3b, formed either by C3(H2O)Bb or by the classical and lectin pathway C3 convertase C4bC2a, binds to the target surfaces and sequesters fB to form C3bfB complexes that, upon cleavage by matCFD, create another C3 convertase complex C3bBb. This convertase can be further stabilized by properdin, which prevents decay of the complex and conversion of C3b by factor H and factor I. C3bBb is the functional convertase of the alternative pathway.

The three pathways converge after formation of the C3 convertases C4bC2a and C3bBb. The C3 cleavage fragment C3a is an anaphylatoxin which promotes inflammation. C3b functions as an opsonin by binding covalently through a thioester bond on the surface of target cells, marking them for circulating complement receptor (CR)-displaying effector cells, such as NK cells and macrophages, which contribute to complement-dependent cellular cytotoxicity (CDCC) and complement-dependent cellular phagocytosis (CDCP), respectively. C3b also binds to the C3 convertase (either C4bC2a or C3bBb) to form a C5 convertase (C4bC2a(C3b)n or C3bBb(C3b)n, respectively), which leads to MAC formation and subsequent CDC. Additionally, C3b's cell-bound degradation fragments, iC3b and C3dg, can promote complement-receptor-mediated cytotoxicity (CDCC and CDCP) as well as adaptive immune response through B cell activation (M. C. Carroll, Nat. Immunol., 5:981, 2004).

Formation of C5 convertase leads to the cleavage of C5 into C5a and C5b. C5a is another anaphylatoxin. C5b recruits C6-9 to form the membrane attack complex (MAC, or C5b-9 complex). The MAC complex causes pore formation resulting in membrane destruction of the target cell and cell lysis (so called complement-dependent cytotoxicity, CDC). Direct cell lysis through the MAC formation has been traditionally recognized as a terminal effector mechanism of the complement system, however, C3b mediated opsonization and pro-inflammatory signaling as well as the anaphylatoxin function of C3a are thought to play a significant role in the mediation of complement dependent inflammatory pathology.

Complement regulatory proteins (CRPs) prevent unwanted complement activation and consumption of complement components. These proteins are present in most cells and via tight control they play an important role in protecting the host cells from complement-mediated damage. CRPs can be soluble proteins (sCRPs) or membrane-bound complement regulatory proteins (mCRPs) (P. F. Zipfel and C. Skerka, Nat. Rev. Immunol. 9:729, 2009). One of the most abundant protease inhibitors in circulating blood is the C1 inhibitor (C1inh), with an average plasma concentration of 0.25 g/L (H. Gregorek, Comp. and Inflamm. 8:310, 1991). C1inh binds to and inactivates C1r, C1s and two of the MBL-associated serine proteases, MASP-1 and MASP-2; hence it is the primary inhibitor for the classical and lectin pathway. Other sCRPs include C4 binding protein (C4BP), and factors H, B, D and I (P. F. Zipfel and C. Skerka, 2009).

In contrast to sCRPs, the mCRPs regulate the complement pathways by targeting both C3 and C4 (P. F. Zipfel and C. Skerka, 2009). For example, CD46 (membrane cofactor protein; MCP) is a co-factor for factor I, which mediates cleavage of C3b and C4b into their inactive degradation products, iC3b and iC4b, respectively, and thereby leads to inhibition of all three complement pathways. CD55 (decay acceleration factor; DAF) accelerates the decay of C3 and C5 convertases, which inhibits all three complement pathways. CD59 (protectin) prevents assembly of the MAC by inhibiting the polymerization of C9 and its subsequent binding to C5b-8, thus inhibiting all three pathways.

While complement activation provides a valuable first line of defense against potential pathogens, the activities of complement that promote a protective immune response can also represent a potential threat to the host (K. R. Kalli, et al., Springer Semin. Immunopathol. 15:417 431, 1994; B. P. Morgan, Eur. J. Clinical Investig. 24:219 228, 1994). For example, C3 and C5 proteolytic products recruit and activate neutrophils. Although indispensable for host defense, activated neutrophils are indiscriminate in their release of destructive enzymes and may cause organ damage. In addition, complement activation may cause the deposition of lytic complement components on nearby host cells as well as on microbial targets, resulting in host cell lysis. Thus, dysregulated and unabated complement activity can also function as a major driver of disease, causing unchecked propagation of inflammation and tissue destruction.

The alternative pathway of complement (AP) is typically described as a downstream amplifier of complement activity, increasing the host immune response following activation of complement via the classical and lectin pathways. However, the ability of the AP to create a positive feedback loop of protease complexes with activity that drives the formation of new complexes of the same type is unique within the complement pathways (Lachmann P. J, Adv Immunol 104:115-49, 2009). Activation of the AP may be a major pathological mechanism in a number of acute and chronic disease states and could represent an effective point for clinical control.

The growing recognition of the importance of complement-mediated tissue injury in a variety of disease states underscores the need for effective complement inhibitory drugs. To date, there are few complement-targeting drugs that have been approved for human use. Eculizumab (Soliris®) and the related molecule ravulizumab (Ultomiris®), are antibodies that selectively bind C5. Avacopan (Tavneos®) is a small-molecule drug that acts as a C5a receptor antagonist and selectively blocks the effects of C5a. Pegcetacoplan (Empaveli®) is a pegylated peptide that binds to and inhibits complement component C3. All of these currently approved drugs inhibit multiple complement pathways, which may result in undesired effects. Therefore, an inhibitor that selectively blocks the initiation steps of a single complement pathway, such as the alternative pathway, would have significant advantages over the existing treatment options.

The role of the complement system in contributing to tissue damage in many clinical conditions, and the lack of targeted treatments that block upstream complement activation, particularly AP activation, highlights the pressing need to develop therapeutically effective complement inhibitors to prevent these adverse effects.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the present disclosure provides methods of treatment using MASP-3 serine protease inhibitors. In some embodiments, the MASP-3 serine protease inhibitors are anti-MASP-3 antibodies or antigen-binding fragments thereof. In some embodiments, the anti-MASP-3 antibodies or antigen-binding fragments thereof comprise a heavy chain variable region comprising an HCDR1, HCDR2, and HCDR3 having the sequences set forth in SEQ ID NO: HCDR1 having the sequences set forth as SEQ ID NO:3, SEQ ID NO:4 or 11, and SEQ ID NO:5, respectively, and a light chain variable region comprising an LCDR1, LCDR2, and LCDR3 having the sequences set forth as SEQ ID NO: 6 or 14, SEQ ID NO:7, and SEQ ID NO:8, respectively.

In some embodiments, the MASP-3 serine protease inhibitors are used in methods of treatment for diseases and disorders related to the alternative pathway of complement. In some embodiments, the disease or disorder is paroxysmal nocturnal hemoglobinuria (PNH). In some embodiments, the disease or disorder is complement 3 glomerulopathy (C3G). In some embodiments, the disease or disorder is idiopathic immune complex-mediated glomerulonephritis (ICGN).

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic diagram of the complement system.

FIG. 2 is a graphical representation of a Phase 1 study of antibody 13B1-10-1-NA in healthy subjects.

FIG. 3 is a graph showing the percent change in mean mature complement factor D (CFD) over time after IV administration of antibody 13B1-10-1-NA to healthy subjects at doses of 3 mg/kg or 5 mg/kg as compared to a placebo.

FIG. 4 is a graphical representation of a Phase 1b study of antibody 13B1-10-1-NA in PNH patients, showing initial adjunctive therapy with of antibody 13B1-10-1-NA and ravulizumab followed by 13B1-10-1-NA monotherapy.

FIG. 5 is a graph showing the mean hemoglobin levels in PNH patients during a Phase 1b study of antibody 13B1-10-1-NA. The patients were adults with confirmed PNH diagnosis who were complement inhibitor treatment-naïve. Antibody 13B1-10-NA was administered at a dose of 5 mg/kg by subcutaneous injection every four weeks. Data shown includes ten patients at differing times following first dosing. The horizontal lines indicate lower limit of normal for males (LLN(M)) and lower limit of normal for females (LLN(F)), as labeled. The number of patients contributing to each data point is shown below the x-axis.

FIG. 6 is a graph showing the hemoglobin levels in each of ten PNH patients during a Phase 1b study of antibody 13B1-10-1-NA. The patients, dosing, and timepoints are as described for FIG. 5. The horizontal lines indicate lower limit of normal for males (LLN(M)) and lower limit of normal for females (LLN(F)), as labeled. Male patients are indicated with squares; female patients are indicated with circles. Patients 6 and 7, indicated with an asterisk, also suffered from myelodysplastic syndrome (MDS).

FIG. 7 is a graph showing the mean LDH levels in PNH patients during a Phase 1b study of antibody 13B1-10-1-NA. The patients, dosing, and timepoints are as described for FIG. 5. The horizontal lines indicate upper limit of normal (ULN) and 1.5× the upper limit of normal (ULN 1.5×), as labeled. The number of patients contributing to each data point is shown below the x-axis.

FIG. 8 is a graph showing the LDL levels in each of ten PNH patients during a Phase 1b study of antibody 13B1-10-1-NA. The patients, dosing, and timepoints are as described for FIG. 5. The horizontal lines indicate upper limit of normal (ULN) and 1.5× the upper limit of normal (ULN 1.5×), as labeled.

FIG. 9 is a graph showing the mean absolute reticulocyte count in PNH patients during a Phase 1b study of antibody 13B1-10-1-NA. The patients, dosing, and timepoints are as described for FIG. 5. The horizontal lines indicate upper limit of normal (ULN) and lower limit of normal (LLN), as labeled. The number of patients contributing to each data point is shown below the x-axis.

FIG. 10 is a graph showing the absolute reticulocyte count in each of ten PNH patients during a Phase 1b study of antibody 13B1-10-1-NA. The patients, dosing, and timepoints are as described for FIG. 5. The horizontal lines indicate upper limit of normal (ULN) and lower limit of normal (LLN), as labeled.

FIG. 11 is a graph showing the mean GPI-deficient red blood cell clone size in PNH patients during a Phase 1b study of antibody 13B1-10-1-NA. The patients, dosing, and timepoints are as described for FIG. 5. The number of patients contributing to each data point is shown below the x-axis.

### DETAILED DESCRIPTION

### I. Definitions

Unless specifically defined herein, all terms used herein have the same meaning as would be understood by those of ordinary skill in the art of the present invention. The following definitions are provided in order to provide clarity with respect to the terms as they are used in the specification and claims to describe the present invention. Additional definitions are set forth throughout this disclosure.

In the present descriptions, any concentration range, percentage range, ratio range, or integer range is to be understood to include the value of any integer within the recited range and, when appropriate, fractions thereof (such as one tenth and one hundredth of an integer), unless otherwise indicated or evident from the context. Any number range recited herein relating to any physical feature, such as polymer subunits, size, or thickness, is to be understood to include any integer within the recited range and, when appropriate, fractions thereof, unless otherwise indicated or evident from the context. As used herein, the term “about” is meant to specify that the range or value provided may vary by ±10% of the indicated range or value, unless otherwise indicated.

It should be understood that the terms “a”, “an”, and “the” as used herein refer to one or more of the referenced components. The use of the alternative (e.g., “or”) should be understood to mean either one, both, or any combination of the alternatives. As used herein, the terms “include”, “have”, and “comprise” are used synonymously, which terms and variants thereof are intended to be construed as non-limiting.

“Optional” or “optionally” means that the subsequently described element, component, event, or circumstance may or may not occur, and that the description includes instances in which the element component, event, or circumstance occurs and instances in which it does not.

It should be understood that the individual constructs or groups of constructs derived from the various combinations of the structures and subunits described herein are disclosed by the present application to the same extent as if each construct or group of constructs was set forth individually. Thus, selection of particular structures or particular subunits is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain) or a protein “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, the terms “treat”, “treatment”, or “ameliorate” refer to medical management of a disease, disorder, or condition of a subject. In general, an appropriate dose or treatment regimen comprising a targeted complement-activating molecule or composition of the present disclosure is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease, stabilization of disease state; delay or prevention of disease progression; remission; survival; prolonged survival; or any combination thereof.

A “therapeutically effective amount” or “effective amount” of a targeted complement-activating molecule, polynucleotide, vector, host cell, or composition of this disclosure refers to an amount of the composition or molecule sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient, administered alone, a therapeutically effective amount refers to the effects of that ingredient or a cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially, sequentially, or simultaneously.

As used herein, “a subject” includes all mammals, including without limitation humans, non-human primates, dogs, cats, horses, sheep, goats, cows, rabbits, pigs, and rodents. A subject may be male or female, and can be any suitable age, including infant, juvenile, adolescent, adult, and geriatric subjects.

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

In the broadest sense, the naturally occurring amino acids can be divided into groups based upon the chemical characteristic of the side chain of the respective amino acids. By “hydrophobic” amino acid is meant either Ile, Leu, Met, Phe, Trp, Tyr, Val, Ala, Cys or Pro. By “hydrophilic” amino acid is meant either Gly, Asn, Gln, Ser, Thr, Asp, Glu, Lys, Arg or His.

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

As used herein, “protein” or “peptide” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid, and non-naturally occurring amino acid polymers. Variants of proteins, peptides, and polypeptides of this disclosure are also contemplated. In certain embodiments, variant proteins, peptides, and polypeptides comprise or consist of an amino acid sequence that is at least 70%, 75%, 80%, 85%, 90%, 91%, 92%, 93%, 94%, 95%, 96%, 97%, 98%, 99%, or 99.9% identical to an amino acid sequence of a defined or reference amino acid sequence as described herein.

“Nucleic acid molecule” or “oligonucleotide” or “polynucleotide” or “polynucleic acid” refers to an oligomeric or polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), which includes, for example, mRNA, microRNA, siRNA, viral genomic RNA, and synthetic RNA, and polydeoxyribonucleic acid (DNA), which includes, for example, cDNA, genomic DNA, and synthetic DNA. Both RNA and DNA may be single or double stranded. If single-stranded, the nucleic acid molecule may be the coding strand or non-coding (anti-sense) strand. A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

Variants of nucleic acid molecules of this disclosure are also contemplated. Variant nucleic acid molecules are at least 70%, 75%, 80%, 85%, 90%, and are preferably 95%, 96%, 97%, 98%, 99%, or 99.9% identical a nucleic acid molecule of a defined or reference polynucleotide as described herein, or that hybridize to a polynucleotide under stringent hybridization conditions of 0.015M sodium chloride, 0.0015M sodium citrate at about 65-68° C. or 0.015M sodium chloride, 0.0015M sodium citrate, and 50% formamide at about 42° C. Nucleic acid molecule variants retain the capacity to encode a binding domain thereof having a functionality described herein, such as binding a target molecule.

“Percent sequence identity” refers to a relationship between two or more sequences, as determined by comparing the sequences. Preferred methods to determine sequence identity are designed to give the best match between the sequences being compared. For example, the sequences are aligned for optimal comparison purposes (e.g., gaps can be introduced in one or both of a first and a second amino acid or nucleic acid sequence for optimal alignment). Further, non-homologous sequences may be disregarded for comparison purposes. The percent sequence identity referenced herein is calculated over the length of the reference sequence, unless indicated otherwise. Methods to determine sequence identity and similarity can be found in publicly available computer programs. Sequence alignments and percent identity calculations may be performed using a BLAST program (e.g., BLAST 2.0, BLASTP, BLASTN, or BLASTX), or Megalign (DNASTAR) software. The mathematical algorithm used in the BLAST programs can be found in Altschul et al., Nucleic Acids Res. 25:3389-3402, 1997. Appropriate parameters for measuring alignment, including any algorithms needed to achieve maximal alignment over the full-length of the sequences being compared can be determined by known methods.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such a nucleic acid could be part of a vector and/or such a nucleic acid or polypeptide could be part of a composition (e.g., a cell lysate), and still be isolated in that such vector or composition is not part of the natural environment for the nucleic acid or polypeptide. “Isolated” can, in some embodiments, also describe an antibody, antigen-binding fragment, polynucleotide, vector, host cell, or composition that is outside of a human body.

The term “gene” means the segment of DNA or RNA involved in producing a polypeptide chain; in certain contexts, it includes regions preceding and following the coding region (e.g., 5′ untranslated region (UTR) and 3′ UTR) as well as intervening sequences (introns) between individual coding segments (exons).

A “functional variant” refers to a polypeptide or polynucleotide that is structurally similar or substantially structurally similar to a parent or reference compound of this disclosure, but differs slightly in composition (e.g., one or more base, atom or functional group is different, added, or removed), such that the polypeptide or encoded polypeptide is capable of performing at least one function of the parent polypeptide with at least 50% efficiency, preferably at least 55%, 60%, 70%, 75%, 80%, 85%, 90%, 95%, 96%, 97%, 98%, 99%, 99.9%, 100% level of activity of the parent polypeptide, or a level of activity greater than that of the parent polypeptide. In other words, a functional variant of a polypeptide or encoded polypeptide of this disclosure has “similar binding,” “similar affinity” or “similar activity” when the functional variant displays an improvement in performance, or no more than a 50% reduction in performance, in a selected assay as compared to the parent or reference polypeptide, such as an assay for measuring enzymatic activity or binding affinity.

As used herein, a “functional portion” or “functional fragment” refers to a polypeptide or polynucleotide that comprises only a domain, portion or fragment of a parent or reference compound, and the polypeptide or encoded polypeptide retains at least 50% activity associated with the domain, portion or fragment of the parent or reference compound, preferably at least 55%, 60%, 70%, 75%, 80%, 85%, 90%, 95%, 96%, 97%, 98%, 99%, 99.9%, 100% level of activity of the parent polypeptide, or a level of activity greater than that of the parent polypeptide, or provides a biological benefit (e.g., effector function). A “functional portion” or “functional fragment” of a polypeptide or encoded polypeptide of this disclosure has “similar binding” or “similar activity” when the functional portion or fragment displays an improvement in performance, or no more than a 50% reduction in performance, in a selected assay as compared to the parent or reference polypeptide (preferably no more than 20% or 10% reduction, or no more than a log difference as compared to the parent or reference with regard to affinity).

As used herein, the term “engineered,” “recombinant,” or “non-natural” refers to an organism, microorganism, cell, protein, polypeptide, nucleic acid molecule, or vector that includes at least one genetic alteration or has been modified by introduction of an exogenous or heterologous nucleic acid molecule, wherein such alterations or modifications are introduced by genetic engineering (i.e., human intervention). Genetic alterations include, for example, modifications introducing expressible nucleic acid molecules encoding functional RNA, proteins, fusion proteins or enzymes, or other nucleic acid molecule additions, deletions, substitutions, or other functional disruption of a cell's genetic material. Additional modifications include, for example, non-coding regulatory regions in which the modifications alter expression of a polynucleotide, gene, or operon.

As used herein, “heterologous” or “non-endogenous” or “exogenous” refers to any gene, protein, compound, nucleic acid molecule, or activity that is not native to a host cell or a subject, or any gene, protein, compound, nucleic acid molecule, or activity native to a host cell or a subject that has been altered. Heterologous, non-endogenous, or exogenous includes genes, proteins, compounds, or nucleic acid molecules that have been mutated or otherwise altered such that the structure, activity, or both is different as between the native and altered genes, proteins, compounds, or nucleic acid molecules. In certain embodiments, heterologous, non-endogenous, or exogenous genes, proteins, or nucleic acid molecules (e.g., receptors, ligands, etc.) may not be endogenous to a host cell or a subject, but instead nucleic acids encoding such genes, proteins, or nucleic acid molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). The term “homologous” or “homolog” refers to a gene, protein, compound, nucleic acid molecule, or activity found in or derived from a host cell, species, or strain. For example, a heterologous or exogenous polynucleotide or gene encoding a polypeptide may be homologous to a native polynucleotide or gene and encode a homologous polypeptide or activity, but the polynucleotide or polypeptide may have an altered structure, sequence, expression level, or any combination thereof. A non-endogenous polynucleotide or gene, as well as the encoded polypeptide or activity, may be from the same species, a different species, or a combination thereof.

In certain embodiments, a nucleic acid molecule or portion thereof native to a host cell will be considered heterologous to the host cell if it has been altered or mutated, or a nucleic acid molecule native to a host cell may be considered heterologous if it has been altered with a heterologous expression control sequence or has been altered with an endogenous expression control sequence not normally associated with the nucleic acid molecule native to a host cell. In addition, the term “heterologous” can refer to a biological activity that is different, altered, or not endogenous to a host cell. As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding an antibody or antigen-binding fragment (or other polypeptide), or any combination thereof.

As used herein, the term “endogenous” or “native” refers to a polynucleotide, gene, protein, compound, molecule, or activity that is normally present in a host cell or a subject.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, posttranslational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a protein (e.g., a heavy chain of an antibody), or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of different encoding nucleic acid molecules or the number of different protein activities, not the number of separate nucleic acid molecules introduced into a host cell.

The term “construct” refers to any polynucleotide that contains a recombinant nucleic acid molecule (or, when the context clearly indicates, a fusion protein of the present disclosure). A (polynucleotide) construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Vectors of the present disclosure also include transposon systems (e.g., Sleeping Beauty, see, e.g., Geurts et al., Mol. Ther. 8:108, 2003: Mátés et al., Nat. Genet. 41:753, 2009). Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, “expression vector” or “vector” refers to a DNA construct containing a nucleic acid molecule that is operably linked to a suitable control sequence capable of effecting the expression of the nucleic acid molecule in a suitable host. Such control sequences typically include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself or deliver the polynucleotide contained in the vector into the genome without the vector sequence. In the present specification, “plasmid,” “expression plasmid,” “virus,” and “vector” are often used interchangeably.

The term “introduced” in the context of inserting a nucleic acid molecule into a cell, means “transfection”, “transformation,” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

In certain embodiments, polynucleotides of the present disclosure may be operatively linked to certain elements of a vector. For example, polynucleotide sequences that are needed to affect the expression and processing of coding sequences to which they are ligated may be operatively linked. Expression control sequences may include appropriate transcription initiation, termination, promoter, and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest may also be considered operatively linked.

In certain embodiments, the vector comprises a plasmid vector or a viral vector (e.g., a lentiviral vector or a γ-retroviral vector). Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox, and canarypox). Other viruses include, for example, Norwalk virus, togavirus, flavivirus, reoviruses, papovavirus, hepadnavirus, and hepatitis virus. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996). Methods of using retroviral and lentiviral viral vectors and packaging cells for transducing mammalian host cells with viral particles containing transgenes are known in the art and have been previous described, for example, in: U.S. Pat. No. 8,119,772; Walchli et al., PLoS One 6:327930, 2011; Zhao et al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors that can be used with the compositions and methods of this disclosure include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as Sleeping Beauty or other transposon vectors).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

Plasmid vectors, including DNA-based plasmid vectors for expression of one or more proteins in vitro or for direct administration to a subject, are also known in the art. Such vectors may comprise a bacterial origin of replication, a viral origin of replication, genes encoding components required for plasmid replication, and/or one or more selection markers, and may also contain additional sequences allowing for bicistronic or multicistronic expression.

As used herein, the term “host” refers to a cell or microorganism targeted for genetic modification with a heterologous nucleic acid molecule to produce a polypeptide of interest (e.g., an antibody of the present disclosure).

A host cell may include any individual cell or cell culture which may receive a vector or the incorporation of nucleic acids or express proteins. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989).

“Antigen”, as used herein, refers to an immunogenic molecule that provokes an immune response. This immune response may involve antibody production, activation of specific immunologically competent cells, activation of complement, antibody dependent cytotoxicity, or any combination thereof. An antigen (immunogenic molecule) may be, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, stool samples, cells, biological fluids, or combinations thereof. Antigens can be produced by cells that have been modified or genetically engineered to express an antigen. Antigens can also be present in or on an infectious agent, such as present in a virion, or expressed or presented on the surface of a cell infected by infectious agent.

The term “epitope” or “antigenic epitope” includes any molecule, structure, amino acid sequence, or protein determinant that is recognized and specifically bound by a cognate binding molecule, such as an immunoglobulin, or other binding molecule, domain, or protein. Epitopic determinants generally contain chemically active surface groupings of molecules, such as amino acids or sugar side chains, and can have specific three-dimensional structural characteristics, as well as specific charge characteristics. Where an antigen is or comprises a peptide or protein, the epitope can be comprised of consecutive amino acids (e.g., a linear epitope), or can be comprised of amino acids from different parts or regions of the protein that are brought into proximity by protein folding (e.g., a discontinuous or conformational epitope), or non-contiguous amino acids that are in close proximity irrespective of protein folding.

The term “antibody” refers to an immunoglobulin molecule consisting of one or more polypeptides that specifically binds an antigen through at least one epitope recognition site. For example, the term “antibody” encompasses an intact antibody comprising at least two heavy chains and two light chains connected by disulfide bonds, as well as any antigen-binding portion or fragment of an intact antibody that has or retains the ability to bind to the antigen target molecule recognized by the intact antibody, such as an scFv, Fab, or Fab′2 fragment. The term also encompasses full-length or fragments of antibodies of any class or sub-class, including IgG and sub-classes thereof (such as IgG1, IgG2, IgG3, and IgG4), IgM, IgE, IgA, and IgD.

The term “antibody” is used herein in the broadest sense, encompassing antibodies and antibody fragments thereof, derived from any antibody producing mammal (e.g., mouse, rat, rabbit, and primate including human), or from a hybridoma, phage selection, recombinant expression or transgenic animals (or other methods of producing antibodies or antibody fragments). It is not intended that the term “antibody” be limited as regards to the source of the antibody or manner in which it is made (e.g., by hybridoma, phage selection, recombinant expression, transgenic animal, peptide synthesis, etc.). Exemplary antibodies include polyclonal, monoclonal and recombinant antibodies; multispecific antibodies (e.g., bispecific antibodies); humanized antibodies; fully human antibodies, murine antibodies; chimeric, mouse human, mouse primate, primate human monoclonal antibodies; and anti-idiotype antibodies, and may be any intact molecule or fragment thereof. As used herein, the term “antibody” encompasses not only intact polyclonal or monoclonal antibodies, but also fragments thereof (such as dAb, Fab, Fab′, F(ab′)2, Fv), single chain (ScFv), synthetic variants thereof, naturally occurring variants, fusion proteins comprising an antibody portion with an antigen-binding fragment of the required specificity, humanized antibodies, chimeric antibodies, and any other modified configuration of the immunoglobulin molecule that comprises an antigen-binding site or fragment (epitope recognition site) of the required specificity. The term encompasses genetically engineered and-or otherwise modified forms of immunoglobulins such as intrabodies, peptibodies, diabodies, triabodies, tetrabodies, tandem di-scFv, tandem tri-scFv, and the like, including antigen-binding fragments thereof.

The terms “VH” and “VL” refer to the variable binding regions from an antibody heavy chain and an antibody light chain, respectively. A VL may be a kappa class chain or a lambda class chain. The variable binding regions comprise discrete, well-defined sub-regions known as complementarity determining regions (CDRs) and framework regions (FRs). The CDRs are located within a hypervariable region (HVR) of the antibody and refer to sequences of amino acids within antibody variable regions which, in general, together confer the antigen specificity and/or binding affinity of the antibody. Consecutive CDRs (i.e., CDR1 and CDR2, and CDR2 and CDR3) are separated from one another in primary structure by a framework region.

As used herein, a “chimeric antibody” is a recombinant protein that contains the variable domains and complementarity determining regions derived from a non-human species (e.g., rodent) antibody, while the remainder of the antibody molecule is derived from a human antibody. In some embodiments, a chimeric antibody is comprised of an antigen-binding fragment of one antibody operably linked or otherwise fused to a heterologous Fc portion of a different antibody. For example, a mouse-human chimeric antibody may comprise an antigen-binding fragment of a mouse antibody fused to an Fc portion derived from a human antibody. In some embodiments, the heterologous Fe domain may be from a different Ig class from the parent antibody, including IgA (including subclasses IgA1 and IgA2), IgD, IgE, IgG (including subclasses IgG1, IgG2, IgG3 and IgG4) and IgM.

As used herein, a “humanized antibody” is a molecule, generally prepared using recombinant techniques, having an antigen-binding site derived from an immunoglobulin from a non-human species and the remaining immunoglobulin structure of the molecule based upon the structure and/or sequence of a human immunoglobulin. A humanized antibody differs from a chimeric antibody in that typically only the CDRs from the non-human species are used, grafted onto appropriate framework regions in a human variable domain. Antigen binding sites may be wild type or may be modified by one or more amino acid substitutions. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, six) which are altered with respect to the original antibody, which are also termed one or more CDRs “derived from” one or more CDRs from the original antibody.

As used herein, the term “antibody fragment” refers to a portion derived from or related to a full-length antibody, generally including the antigen binding or variable region thereof. Illustrative examples of antibody fragments include Fab, Fab′, F(ab)2, F(ab′)2 and Fv fragments, scFv fragments, diabodies, linear antibodies, single chain antibody molecules and multispecific antibodies formed from antibody fragments.

As used herein, the term “antigen-binding fragment” refers to a polypeptide fragment that contains at least one CDR of an immunoglobulin heavy and/or light chains that specifically binds to the antigen to which the antibody was raised. An antigen-binding fragment may comprise 1, 2, 3, 4, 5, or all 6 CDRs of a VH and VL sequence from an antibody.

A “Fab” (fragment antigen binding) is the part of an antibody that binds to antigens and includes the variable region and CH1 of the heavy chain linked to the light chain via an inter-chain disulfide bond. Each Fab fragment is monovalent with respect to antigen binding, i.e., it has a single antigen-binding site. Pepsin treatment of an antibody yields a single large F(ab′)2 fragment that roughly corresponds to two disulfide-linked Fab fragments having divalent antigen-binding activity and is still capable of cross-linking antigen. Both the Fab and F(ab′)2 are examples of “antigen-binding fragments.” Fab′ fragments differ from Fab fragments by having a few additional residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments are often produced as pairs of Fab′ fragments that have hinge cysteines between them. Other chemical couplings of antibody fragments are also known.

Fab fragments may be joined, e.g., by a peptide linker, to form a single chain Fab, also referred to herein as “scFab.” In these embodiments, an inter-chain disulfide bond that is present in a native Fab may not be present, and the linker serves in full or in part to link or connect the Fab fragments in a single polypeptide chain. A heavy-chain derived Fab fragment (e.g., comprising, consisting of, or consisting essentially of VH+CH1, or “Fd”) and a light chain-derived Fab fragment (e.g., comprising, consisting of, or consisting essentially of VL+CL) may be linked in any arrangement to form a scFab. For example, a scFab may be arranged, in N-terminal to C-terminal direction, according to (heavy chain Fab fragment-linker-light chain Fab fragment) or (light chain Fab fragment-linker-heavy chain Fab fragment).

“Fv” is a small antibody fragment that contains a complete antigen-recognition and antigen-binding site. This fragment generally consists of a dimer of one heavy- and one light-chain variable region domain in tight, non-covalent association. However, even a single variable domain (or half of an Fv comprising only three CDRs specific for an antigen) has the ability to recognize and bind antigen, although typically at a lower affinity than the entire binding site.

“Single-chain Fv” also abbreviated as “sFv” or “scFv”, are antibody fragments that comprise the VH and VL antibody domains connected into a single polypeptide chain. The scFv polypeptide may comprise a polypeptide linker disposed between and linking the VH and VL domains that enables the scFv to retain or form the desired structure for antigen binding, although a linker is not always required. Such a peptide linker can be incorporated into a fusion polypeptide using standard techniques well known in the art. Additionally, or alternatively, Fv can have a disulfide bond formed between and stabilizing the VH and the VL. For a review of scFv, see Pluckthun in The Pharmacology of Monoclonal Antibodies, vol. 113, Rosenburg and Moore eds., Springer-Verlag, New York, pp. 269-315 (1994). In certain embodiments, the antibody or antigen-binding fragment comprises a scFv comprising a VH domain, a VL domain, and a peptide linker linking the VH domain to the VL domain. In particular embodiments, a scFv comprises a VH domain linked to a VL domain by a peptide linker, which can be in a VH-linker-VL orientation or in a VLlinker-VH orientation. Any scFv of the present disclosure may be engineered so that the C-terminal end of the VL domain is linked by a short peptide sequence to the N-terminal end of the VH domain, or vice versa (i.e., (N)VL(C)-linker-(N)VH(C) or (N)VH(C)-linker-(N)VL(C)). Alternatively, in some embodiments, a linker may be linked to an N-terminal portion or end of the VH domain, the VL domain, or both.

Peptide linker sequences for use in scFv or in other fusion proteins, such as the targeted complement-activating molecules described herein, may be chosen, for example, based on: (1) their ability to adopt a flexible extended conformation; (2) their inability or lack of ability to adopt a secondary structure that could interact with functional epitopes on the first and second polypeptides and/or on a target molecule; and/or (3) the lack or relative lack of hydrophobic or charged residues that might react with the polypeptides and/or target molecule. Other considerations regarding linker design (e.g., length) can include the conformation or range of conformations in which the VH and VL can form a functional antigen-binding site. In certain embodiments, peptide linker sequences contain, for example, Gly, Asn and Ser residues. Other near neutral amino acids, such as Thr and Ala, may also be included in a linker sequence. Other amino acid sequences which may be usefully employed as linker include those disclosed in Maratea et al., Gene 40:39 46 (1985); Murphy et al., Proc. Natl. Acad. Sci. USA 83:8258 8262 (1986); U.S. Pat. Nos. 4,935,233, and 4,751,180. Other illustrative and non-limiting examples of linkers may include, for example, the pentamer Gly-Gly-Gly-Gly-Ser (GGGGS; SEQ ID NO:18) when present in a single iteration or repeated one to five times or more, and may begin or end in a partial iteration, such as, for example, GGGGSGGGGSGGGG (SEQ ID NO:19). Any suitable linker may be used, and in general can be about 3, 4, 5, 6, 7, 8, 9, 10, 11, 12, 13, 14, 15, 16, 17, 18, 19, 20, 21, 22, 15 23, 24, 25, 26, 27, 28, 29, 30, 40, 50, 60, 70, 80, 90, 100 amino acids in length, or less than about 200 amino acids in length, and will preferably comprise a flexible structure (can provide flexibility and room for conformational movement between two regions, domains, motifs, fragments, or modules connected by the linker), and will preferably be biologically inert and/or have a low risk of immunogenicity in a human.

Antibodies may be monospecific (e.g., binding to a single epitope) or multispecific (e.g., binding to multiple epitopes and/or target molecules). A bispecific or multispecific antibody or antigen-binding fragment may, in some embodiments, comprise one, two, or more antigen-binding domains (e.g., a VH and a VL). Two or more binding domains may be present that bind to the same or different epitopes, and a bispecific or multispecific antibody or antigen-binding fragment as provided herein can, in some embodiments, two or more binding domains, that bind to different antigens or pathogens altogether.

Antibodies and antigen-binding fragments may be constructed in various formats. Exemplary antibody formats disclosed in Spiess et al., Mol. Immunol. 67(2):95 (2015), and in Brinkmann and Kontermann, mAbs 9(2):182-212 (2017), which formats and methods of making the same are incorporated herein by reference and include, for example, Bispecific T cell Engagers (BiTEs), DARTs, Knobs-Into-Holes (KIH) assemblies, scFv-CH3-KIH assemblies, KIH Common Light-Chain antibodies, TandAbs, Triple Bodies, TriBi Minibodies, Fab-scFv, scFv-CH-CL-scFv, F(ab′)2-scFv2, tetravalent HCabs, Intrabodies, CrossMabs, Dual Action Fabs (DAFs) (two-in-one or four-in-one), DutaMabs, DT-IgG, Charge Pairs, Fab-arm Exchange, SEEDbodies, Triomabs, LUZ-Y assemblies, Fcabs, κλ-bodies, orthogonal Fabs, DVD-Igs (e.g., U.S. Pat. No. 8,258,268, which formats are incorporated herein by reference in their entirety), IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, and DVI-IgG (four-in-one), as well as so-called FIT-Ig (e.g., PCT 5 Publication No. WO 2015/103072, which formats are incorporated herein by reference in their entirety), so-called WuxiBody formats (e.g., PCT Publication No. WO 2019/057122, which formats are incorporated herein by reference in their entirety), and so-called In-Elbow-Insert Ig formats (IEI-Ig; e.g., PCT Publication Nos. WO 2019/024979 and WO 2019/025391, which formats are incorporated herein by reference in their entirety).

An antibody or antigen-binding fragment may comprise two or more VH domains, two or more VL domains, or both (i.e., two or more VH domains and two or more VL domains). In particular embodiments, an antigen-binding fragment comprises the format (N-terminal to C-terminal direction) VH-linker-VL-linker-VH-linker-VL, wherein the two VH sequences can be the same or different and the two VL sequences can be the same or different. Such linked scFvs can include any combination of VH and VL domains arranged to bind to a given target, and in formats comprising two or more VH and/or two or more VL, one, two, or more different epitopes or antigens may be bound. It will be appreciated that formats incorporating multiple antigen-binding domains may include VH and/or VL sequences in any combination or orientation. For example, the antigen-binding fragment can comprise the format VL-linker-VH-linker-VL-linker-VH, VH-linker-VL-linker-VL-linker-VH, or VL-linker-VH-linker-VH-linker-VL.

As used herein, the modifier “monoclonal” indicates the character of the antibody as being obtained from a substantially homogenous population of antibodies, and is not intended to be limited as regards the source of the antibody or the manner in which it is made (e.g., by hybridoma, phage selection, recombinant expression, transgenic animals, etc.). The term “monoclonal antibody” encompasses not only intact monoclonal antibodies and full-length monoclonal antibodies, but also fragments thereof (such as Fab, Fab′, F(ab′)2, Fv), single chain (ScFv), variants thereof, fusion proteins comprising an antigen-binding portion, humanized monoclonal antibodies, chimeric monoclonal antibodies, and any other modified configuration of the immunoglobulin molecule that comprises an antigen-binding fragment (epitope recognition site) of the required specificity and the ability to bind to an epitope. Monoclonal antibodies can be obtained using any technique that provides for the production of antibody molecules by continuous cell lines in culture, such as the hybridoma method described by Kohler, G., et al., Nature 256:495, 1975, or they may be made by recombinant DNA methods (see, e.g., U.S. Pat. No. 4,816,567 to Cabilly). Monoclonal antibodies may also be isolated from phage antibody libraries using the techniques described in Clackson, T., et al., Nature 352:624 628, 1991, and Marks, J. D., et al., J. Mol. Biol. 222:581 597, 1991. Such antibodies can be of any immunoglobulin class including IgG, IgM, IgE, IgA, IgD and any subclass thereof.

The recognized immunoglobulin polypeptides include the kappa and lambda light chains and the alpha, gamma (IgG1, IgG2, IgG3, IgG4), delta, epsilon and mu heavy chains, or equivalents in other species. Full-length immunoglobulin “light chains” (of about 25 kDa or about 214 amino acids) comprise a variable region of about 110 amino acids at the NH2 terminus and a kappa or lambda constant region at the COOH-terminus. Full-length immunoglobulin “heavy chains” (of about 50 kDa or about 446 amino acids) similarly comprise a variable region (of about 116 amino acids) and one of the aforementioned heavy chain constant regions, e.g., gamma (of about 330 amino acids).

The basic four-chain antibody unit is a heterotetrameric glycoprotein composed of two identical light (L) chains and two identical heavy (H) chains. An IgM antibody differs from this plan in that it consists of five of the basic heterotetramer units along with an additional polypeptide called the J chain, and therefore contains 10 antigen binding sites. Secreted IgA antibodies also differ from the basic structure in that they can polymerize to form polyvalent assemblages comprising two to five of the basic four-chain units along with a J chain. Each L chain is linked to an H chain by one covalent disulfide bond, while the two H chains are linked to each other by one or more by one or more disulfide bonds, depending on the H chain isotype. Each H and L chain also has regularly spaced intrachain disulfide bridges. The pairing of a VH and VL together forms a single antigen-binding site.

Each H chain has, at the N-terminus, a variable domain (VH) followed by three constant domains (CH1, CH2, CH3), in the case of alpha, gamma, and delta chains, or four CH domains (CH1, CH2, CH3, CH4), in the case of mu and epsilon chains.

Each L chain has, at the N-terminus, a variable domain (VL) followed by a constant domain (CL) at its other end. When an L chain and an H chain are paired, the VL is aligned with the VH and the CL is aligned with the first constant domain of the heavy chain (CH1). The L chain from any vertebrate species can be assigned to one of two types, called kappa (κ) and lambda (λ), based on the amino acid sequences of their constant domains (CL).

Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated alpha (α), delta (δ), epsilon (ε), gamma (γ) and mu (μ), respectively. The γ and α classes are further divided into subclasses on the basis of minor differences in CH sequence and function, for example, humans express the following subclasses: IgG1, IgG2, IgG3, IgG4, IgA1 and IgA2.

For the structure and properties of the different classes of antibodies, see, e.g., Basic and Clinical Immunology, 8th Edition, Daniel P. Stites, Abba I. Terr and Tristram G. Parslow (eds); Appleton and Lange, Norwalk, Conn., 1994, page 71 and Chapter 6.

The term “variable” refers to that fact that certain segments of the V domains differ extensively in sequence among antibodies. The V domain mediates antigen binding and defines specificity of a particular antibody for its particular antigen. However, the variability is not evenly distributed across the 110 amino acid span of the variable domains. Rather, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a beta-sheet configuration, connected by three hypervariable regions, which form loops connecting, and in some cases forming part of, the n-sheet structure. The hypervariable regions in each chain are held together in close proximity by the FRs and, with the hypervariable regions from the other chain, contribute to the formation of the antigen-binding site of antibodies (see Kabat, et al., Sequences of Proteins of Immunological Interest, 5th Ed. Public Health Service, National Institutes of Health, Bethesda, Md (1991)). The constant domains are not involved directly in binding an antibody to an antigen, but exhibit various effector functions.

As used herein, “effector functions” refer to those biological activities attributable to the Fc region of an antibody. Examples of antibody effector functions include participation in antibody-dependent cellular cytotoxicity (ADCC), C1q binding and complement-dependent cytotoxicity, Fc receptor binding, phagocytosis, down-regulation of cell surface receptors, and B cell activation. Modifications such as amino acid substitutions may be made to an Fc domain in order to modify (e.g., enhance or reduce) one or more functions of an Fc-containing polypeptide. Such functions include, for example, Fc receptor binding, antibody half-life modulation, ADCC function, protein A binding, protein G binding, and complement binding. Amino acid modifications that modify Fc functions include, for example, T250Q/M428L, M252Y/S254T/T256E, H433K/N434F, M428L/N434S, E233P/L234V/L235A/G236Δ/A327G/A330S/P331S, E333A, S239D/A330L/I332E, P257I/Q311, K326W/E333S, S239D/I332E/G236A, N297Q, K322A, S228P, L235E/E318A/K320A/K322A, L234A/L235A, and L234A/L235A/P329G mutations. Other Fc modifications and their effect on Fc function are known in the art.

As used herein, the term “hypervariable region” refers to the amino acid residues of an antibody that are responsible for antigen binding. The hypervariable region contains several “complementarity determining regions” (CDRs). The heavy chain comprises three CDR sequences (CDRH1, CDRH2, and CDRH3) and the light chain comprises three CDR sequences (CDRL1, CDRL2, and CDRL3). A variety of systems exist for identifying and numbering the amino acids that make up the CDRs. For example, the hypervariable region generally comprises CDRs at around about residues 24-34 (L1), 50-56 (L2) and 89-97 (L3) in the light chain variable domain, and at around about 31-35 (H1), 50-65 (H2) and 95-102 (H3) in the heavy chain variable domain when numbering in accordance with the Kabat numbering system as described in Kabat, et al., Sequences of Proteins of Immunological Interest, 5th Ed. Public Health Service, National Institutes of Health, Bethesda, Md (1991); and/or at about residues 24-34 (L1), 50-56 (L2) and 89-97 (L3) in the light chain variable domain, and 26-32 (H1), 52-56 (H2) and 95-102 (H3) in the heavy chain variable domain when numbered in accordance with the Chothia numbering system, as described in Chothia and Lesk, J. Mol. Biol. 196:901-917 (1987); and/or at about residues 27-38 (L1), 56-65 (L2) and 105-117 (L3) in the VL, and 27-38 (H1), 56-65 (H2), and 105-117 (H3) in the VH when numbered in accordance with the IMGT numbering system as described in Lefranc, J. P., et al., Nucleic Acids Res 27:209-212; Ruiz, M., et al., Nucleic Acids Res 28:219-221 (2000). Equivalent residue positions can be annotated and compared for different molecules using Antigen receptor Numbering And Receptor Classification (ANARCI) software tool (2016, Bioinformatics 15:298-300). Accordingly, identification of CDRs of an exemplary variable domain (VH or VL) sequence as provided herein according to one numbering scheme is not exclusive of an antibody comprising CDRs of the same variable domain as determined using a different numbering scheme.

As used herein, “specifically binds” refers to an antibody or antigen-binding fragment that binds to an antigen with a particular affinity, while not significantly associating or uniting with any other molecules or components in a sample. Affinity may be defined as an equilibrium association constant (Ka), calculated as the ratio of kon/koff, with units of 1/M or as an equilibrium dissociation constant Kd), calculated as the ratio of koff/kon with units of M.

In some contexts, antibody and antigen-binding fragments may be described with reference to affinity and/or to avidity for antigen. Unless otherwise indicated, avidity refers to the total binding strength of an antibody or antigen-binding fragment thereof to antigen, and reflects binding affinity, valency of the antibody or antigen-binding fragment (e.g., whether the antibody or antigen-binding fragment comprises one, two, three, four, five, six, seven, eight, nine, ten, or more binding sites), and, for example, whether another agent is present that can affect the binding (e.g., a non-competitive inhibitor of the antibody or antigen-binding fragment).

Each embodiment in this specification is to be applied mutatis mutandis to every other embodiment unless expressly stated otherwise. It is contemplated that any embodiment discussed in this specification can be implemented with respect to any method, kit, reagent, or composition of the invention, and vice versa. Furthermore, compositions of the invention can be used to achieve methods of the invention.

### II. Overview

The alternative pathway of the complement system has been implicated in the pathogenesis of numerous acute and chronic disease states, including paroxysmal nocturnal hemoglobinuria (PNH), complement 3 glomerulopathy (C3G), and idiopathic immune complex-mediated glomerulonephritis (ICGN). The present disclosure describes the use of alternative pathway inhibitors, particularly MASP-3 inhibitors, for treatment of these alternative pathway-related diseases.

### A. The Role of MASP-3 in the Complement System

Mannan-binding lectin-associated serine protease-3 (MASP-3) is an activator of the alternative pathway of complement (AP). MASP-3 is one of three possible products of the MASP1 gene. The primary transcript of MASP1 can be spliced to form an mRNA that encodes MASP-1, MASP-3, or MAp44. Interestingly, each of these three gene products has a distinct activity. MASP-1 is a component of the lectin pathway of the complement system, MAp44 is a nonproteolytic protein, and MASP-3 is an activator of the AP. The MASP-1 and MASP-3 proteins share five structural domains in the N-terminal region but have unique serine protease domains at the C-terminal end (Ammitzboll et al., PLos One 8(9):e73317, 2013). The amino acid sequence of human MASP-3, including the 19 amino acid leader sequence, is provided as SEQ ID NO:17. The serine protease domain of human MASP-3 comprises amino acids 450-728 of SEQ ID NO:17.

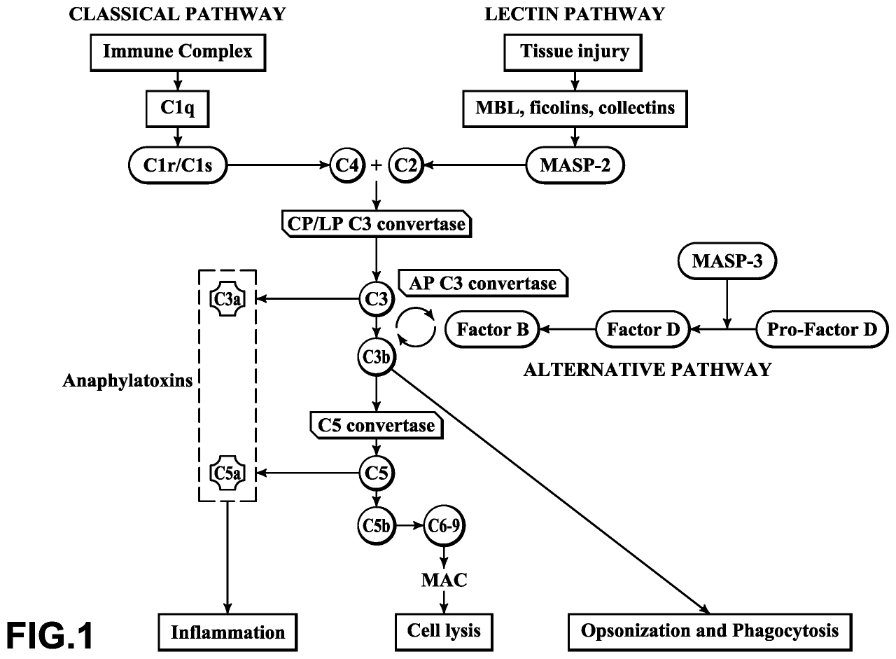

One of the earliest upstream steps in the activation of the AP is the conversion of complement factor D (CFD) from an inactive zymogen to a cleaved or mature form with serine protease activity (Dobo et al., Sci Rep 6:31877, 2016; Oroszlan et al., J Immunol 162(2):857, 2016). See FIG. 1. MASP-3 is responsible for the conversion of CFD from the zymogen to the mature form, thus placing the MASP-3 protein in control of a key upstream regulatory step for the AP (WO2018/026722). Due to the role of MASP-3 at an early stage of AP activation, inhibition of MASP-3 has the potential to provide efficient and targeted inhibition of AP activation.

### B. Paroxysmal Nocturnal Hemoglobinuria

Paroxysmal nocturnal hemoglobinuria (PNH) is an acquired disorder characterized by hemolytic anemia that is driven by uncontrolled activity of the AP on red blood cells (RBCs). Hemolysis results from spontaneous loss of the complement regulatory proteins CD55 and CD59 on a clonally derived subset of RBCs, caused by a global deficiency in all cell surface proteins that are attached by a GPI anchor. If left untreated, PNH is associated with debilitating anemia, a high risk of thrombosis, and a severely reduced survival rate (Risitano et al., Front Immunol. 10:1157, 2019).

Eculizumab and its second-generation variant ravulizumab block terminal complement activation by binding and preventing the cleavage of complement C5. Both mAbs are approved in the United States for treatment of patients with PNH, and they are effective in reducing hemolysis and reducing the risk for thrombosis and death. However, hemolysis is not completely blocked by targeting C5, and enduring anemia persists in most patients. Patients experience low hemoglobin levels and fatigue, and 25%-50% of individuals still require transfusion (Al-Ani et al., Therapeutics and clinical risk management 12:1161, 2016). Incomplete therapeutic benefit of a C5 inhibitor is caused by the extravascular hemolytic pathway in which C3b-opsonized RBCs are destroyed by phagocytes. In fact, extravascular clearance is exacerbated in patients treated with a C5 blocking mAb, as non-lysed PNH RBCs serve as targets for continuous C3b deposition until they are destroyed by phagocytes found in the spleen (Berentsen et al., Ther Adv Hematol 10:2040620719873321, 2019). In some early cases, drastic measures such as splenectomy have been used to ameliorate this condition, but this is not considered standard treatment. (Risitano et al., 2019). Thus, C3b-mediated extravascular hemolysis represents a concern in PNH treatment with terminal pathway inhibitors.

More recently, pegcetacoplan, a pegylated peptide that binds C3 and blocks the enzymatic cleavage of C3 by convertases from the three pathways of complement, was shown to be superior to eculizumab in broad hemolysis control in PNH patients (Hillmen et al., N Engl J Med 384:1028, 2021). Pegcetacoplan treatment improved hemoglobin and other hematologic measures, consistent with inhibition of extravascular hemolysis as well as intravascular hemolysis. However, pegcetacoplan is administered as a twice weekly subcutaneous infusion, which is relatively burdensome for patients.

Thus, a need remains for an effective and convenient treatment for PNH. A proximal complement inhibitor, such as a MASP-3 inhibitor, could provide an improved patient experience, while blocking both intravascular and extravascular hemolysis by inhibition of only the alternative pathway, leaving the classical and lectin pathways intact.

### C. Complement 3 Glomerulopathy

Complement 3 glomerulopathy (C3G) is a disorder related to dysregulation of the AP in plasma and the glomerular microenvironment. Hyperactivation of the AP results in the deposition of C3 and its cleavage products within the glomeruli, leading to inflammation and progressive kidney disease (Smith et al., Nat Rev Nephrol 15:129, 2019; Nephrol Dial Transpl 32(3):459, 2017). AP hyperactivation may have varied causes among patients, including underlying genetic abnormalities of complement genes or auto-antibodies against complement components (Iatropoulos et al., Mol Immunol 71:131, 2016; Corvillo et al., Front Immunol 10:886, 2019). Untreated, the clinical manifestations of C3G vary from proteinuria with relatively preserved kidney function to rapidly progressing kidney failure. Although the disease may remain stable for several years despite persistent proteinuria, almost half of patients reach end-stage kidney disease within five years from clinical diagnosis (Bomback et al., Kidney Int 93(4):977, 2018)

While some treatments are available to help manage symptoms, there are currently no approved medications for C3G. Eculizumab, an anti-C5 monoclonal antibody, has been tested for treatment of C3G, but the response has been shown to be highly heterogeneous. In one study, for example, only three of ten patients obtained a significant reduction in 24-hour proteinuria, suggesting the upstream components of the complement pathways may play a role in C3G (Ruggenenti et al., Am J Kidney Dis 74(2):224, 2019). Thus, a need remains for improved treatments for C3G patients.

### D. Idiopathic Immune Complex-Mediated Glomerulonephritis

Idiopathic immune complex-mediated glomerulonephritis (ICGN), also sometimes referred to as immune complex membranoproliferative glomerulonephritis (IC-MPGN), has similar symptoms to C3G, but different etiology. In both diseases, hyperactivation of the complement system causes damage to the glomerulus, however, in ICGN the initiating event is deposition of immune complexes, which then trigger complement activation. In some cases, this immune complex deposition appears to be related to mutations in complement component proteins (Iatropoulos et al., 2016). ICGN is a progressive disease, with approximately 50% of patients reaching end-stage renal disease within ten years.

While some treatments are available to help manage symptoms, there are currently no approved medications for ICGN. Generalized immunosuppressive treatments have been proposed, but carry the risk of significant side effects.

### III. Antibodies and Antigen-Binding Fragments

Antibodies to MASP-3 have been previously described, including a variety of high-affinity antibodies with serine protease inhibitory activity. See PCT patent publications WO2013/180834, WO2013/192240, and WO2018/026722, which are incorporated herein by reference.

Antibodies described in WO2018/026722 are of particular interest in therapeutic applications, including the antibodies referred to as 13B1, 10D12, 35C1, 4D5, 1F3, 4B6, and 1A10, along with variants and modified versions of these antibodies. A number of such variants are described in WO2018/026722, but additional variants comprising the same or similar CDR sequences could be constructed by one of skill in the art, and such additional variants are also contemplated for use as described herein. Sequences for certain antibodies and variants thereof are provided in the sequence listing in TABLE 1.

It is also contemplated that an antigen-binding fragment of a high-affinity antibody with MASP-3 serine protease inhibitory activity may be used for therapeutic purposes as described herein. Such fragments are known in the art and include single chain antibodies, ScFv, Fab fragments, Fab′ fragments, F(ab′)2 fragments, and univalent antibodies lacking a hinge region.

In some embodiments, the antibody or antigen-binding fragment thereof comprises an HCDR1 having the sequence set forth as SEQ ID NO:3. In some embodiments, the antibody or antigen-binding fragment thereof comprises an HCDR2 having the sequence set forth as SEQ ID NO:4 or 11. In some embodiments, the antibody or antigen-binding fragment thereof comprises an HCDR3 having the sequence set forth as SEQ ID NO:5. In some embodiments, the antibody or antigen-binding fragment thereof comprises an LCDR1 having the sequence set forth as SEQ ID NO: 6 or 14. In some embodiments, the antibody or antigen-binding fragment thereof comprises an LCDR2 having the sequence set forth as SEQ ID NO:7. In some embodiments, the antibody or antigen-binding fragment thereof comprises an LCDR3 having the sequence set forth as SEQ ID NO:8.

In some embodiments, the antibody or antigen-binding fragment thereof comprises an HCDR1 having the sequence set forth as SEQ ID NO:3, an HCDR2 having the sequence set forth as SEQ ID NO:4, an HCDR3 having the sequence set forth as SEQ ID NO:5, an LCDR1 having the sequence set forth as SEQ ID NO:6, an LCDR2 having the sequence set forth as SEQ ID NO:7, and an LCDR3 having the sequence set forth as SEQ ID NO:8. In some embodiments, the antibody or antigen-binding fragment thereof comprises a VH having the sequence set forth as SEQ ID NO:1 and a VL having the sequence set forth as SEQ ID NO:2. In some embodiments, the antibody or antigen-binding fragment thereof is antibody 13B1. In some embodiments, the antibody or antigen-binding fragment thereof comprises a VH having the sequence set forth as SEQ ID NO:12 and a VL having the sequence set forth as SEQ ID NO:10. In some embodiments, the antibody or antigen-binding fragment thereof is antibody 13B1-10-1.

In some embodiments, the antibody or antigen-binding fragment thereof comprises an HCDR1 having the sequence set forth as SEQ ID NO:3, an HCDR2 having the sequence set forth as SEQ ID NO: 11, an HCDR3 having the sequence set forth as SEQ ID NO:5, an LCDR1 having the sequence set forth as SEQ ID NO:6, an LCDR2 having the sequence set forth as SEQ ID NO:7, and an LCDR3 having the sequence set forth as SEQ ID NO:8. In some embodiments, the antibody or antigen-binding fragment thereof comprises a VH having the sequence set forth as SEQ ID NO:9 and a VL having the sequence set forth as SEQ ID NO:10. In some embodiments, the antibody or antigen-binding fragment thereof is antibody 13B1-9-1.

In some embodiments, the antibody or antigen-binding fragment thereof comprises an HCDR1 having the sequence set forth as SEQ ID NO:3, an HCDR2 having the sequence set forth as SEQ ID NO:11, an HCDR3 having the sequence set forth as SEQ ID NO:5, an LCDR1 having the sequence set forth as SEQ ID NO:14, an LCDR2 having the sequence set forth as SEQ ID NO:7, and an LCDR3 having the sequence set forth as SEQ ID NO:8. In some embodiments, the antibody or antigen-binding fragment thereof comprises a VH having the sequence set forth as SEQ ID NO:9 and a VL having the sequence set forth as SEQ ID NO:13. In some embodiments, the antibody or antigen-binding fragment thereof is antibody 13B1-9-1-NA.

In some embodiments, the antibody or antigen-binding fragment thereof comprises an HCDR1 having the sequence set forth as SEQ ID NO:3, an HCDR2 having the sequence set forth as SEQ ID NO:4, an HCDR3 having the sequence set forth as SEQ ID NO:5, an LCDR1 having the sequence set forth as SEQ ID NO:14, an LCDR2 having the sequence set forth as SEQ ID NO:7, and an LCDR3 having the sequence set forth as SEQ ID NO:8. In some embodiments, the antibody or antigen-binding fragment thereof comprises a VH having the sequence set forth as SEQ ID NO: 12 and a VL having the sequence set forth as SEQ ID NO:13. In some embodiments, the antibody or antigen-binding fragment thereof comprises a light chain having the sequence set forth as SEQ ID NO:15 and a heavy chain having the sequence set forth as SEQ ID NO:16. In some embodiments, the antibody or antigen-binding fragment thereof is antibody 13B1-10-1-NA.

### IV. Pharmaceutical Compositions

The MASP-3 antibodies described above may be incorporated into compositions that comprise one or more pharmaceutically acceptable carriers, excipients, or diluents.

A pharmaceutically acceptable carrier is non-toxic, biocompatible and is selected so as not to detrimentally affect the biological activity of the therapeutic agent (and any other therapeutic agents combined therewith). Examples of pharmaceutically acceptable carriers for peptides are described in U.S. Pat. No. 5,211,657 to Yamada. The therapeutic agents described herein may be formulated into preparations in solid, semi solid, gel, liquid or gaseous forms such as tablets, capsules, powders, granules, ointments, solutions, depositories, inhalants and injections allowing for oral, parenteral or surgical administration. Local administration of the compositions by coating medical devices and the like is also contemplated.

Suitable carriers for parenteral delivery via injection, infusion, irrigation or topical delivery include distilled water, physiological phosphate buffered saline, normal or lactated Ringer's solutions, dextrose solution, Hank's solution, or propanediol. In addition, sterile, fixed oils may be employed as a solvent or suspending medium. For this purpose, any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting example, microparticles, microspheres, nanospheres or nanoparticles composed of proteins, liposomes, carbohydrates, synthetic organic compounds, inorganic compounds, polymeric or copolymeric hydrogels and polymeric micelles. Suitable hydrogel and micelle delivery systems include the PEO:PHB:PEO copolymers and copolymer/cyclodextrin complexes disclosed in WO 2004/009664 A2 and the PEO and PEO/cyclodextrin complexes disclosed in U.S. Patent Application Publication No. 2002/0019369 A1. Such hydrogels may be injected locally at the site of intended action, or subcutaneously or intramuscularly to form a sustained release depot.

Compositions of the present invention may be formulated for delivery by any appropriate method including, without limitation, oral, topical, transdermal, sublingual, buccal, subcutaneously, intra-muscularly, intravenously, intra-arterially or as an inhalant.

The compositions of the present invention may also include biocompatible excipients, such as dispersing or wetting agents, suspending agents, diluents, buffers, penetration enhancers, emulsifiers, binders, thickeners, flavoring agents (for oral administration).

Pharmaceutical compositions according to certain embodiments of the present invention are formulated so as to allow the active ingredients contained therein to be bioavailable upon administration of the composition to a patient. Compositions that will be administered to a subject may take the form of one or more dosage units, and a container of a herein described therapeutic agent may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain an effective amount of therapeutic agent or composition of the present disclosure, for treatment of a disease or condition of interest in accordance with teachings herein.

A composition may be in the form of a solid or liquid. In some embodiments, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like. Such a solid composition will typically contain one or more inert fillers or diluents such as sucrose, cornstarch, or cellulose. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the composition is in the form of a capsule, for example, a gelatin capsule, it may contain, in addition to materials of the above type, a liquid carrier such as polyethylene glycol or oil.

The composition may be in the form of a liquid, for example, an elixir, syrup, solution, emulsion or suspension. The liquid may be for oral administration or for delivery by injection, as two examples. When intended for oral administration, preferred compositions contain, in addition to the present compounds, one or more of a sweetening agent, preservative, dye/colorant and flavor enhancer. In a composition intended to be administered by injection, one or more of a surfactant, preservative, wetting agent, dispersing agent, suspending agent, buffer, stabilizer and isotonic agent may be included.

Liquid pharmaceutical compositions, whether they be solutions, suspensions or other like form, may include one or more of the following excipients: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred excipient. An injectable pharmaceutical composition is preferably sterile.

A liquid composition intended for either parenteral or oral administration should contain an amount of a therapeutic agent as described herein such that a suitable dosage will be obtained. The term “parenteral” includes subcutaneous, intravenous, intramuscular, intrasternal, or intra-arterial injection or infusion. Typically, the therapeutic agent is at least 0.01% of the composition. When intended for oral administration, this amount may be varied to be between about 0.1% and about 70% of the weight of the composition. Certain oral pharmaceutical compositions contain between about 4% and about 75% therapeutic agent.

The composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a composition for topical administration. If intended for transdermal administration, the composition may include a transdermal patch or iontophoresis device. The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

A composition may include various materials which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The composition in solid or liquid form may include an agent that binds to the therapeutic agent(s) of the disclosure and thereby assists in the delivery of the compound. Suitable agents that may act in this capacity include one or more proteins or a liposome.

The composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi-phasic, or tri-phasic system in order to deliver the active ingredient(s). Delivery of the aerosol includes the necessary container, activators, valves, sub-containers, and the like, which together may form a kit. One of ordinary skill in the art, without undue experimentation, may determine preferred aerosols.

The pharmaceutical compositions may be prepared by methodology well known in the pharmaceutical art. For example, a composition intended to be administered by injection can be prepared by combining a composition that comprises therapeutic agent as described herein and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the composition so as to facilitate dissolution or homogeneous suspension in the aqueous delivery system.

The pharmaceutical composition may comprise a MASP-3 inhibitory antibody, or antigen-binding fragment thereof, in an aqueous solution. In some embodiments, the pharmaceutical composition comprises a MASP-3 inhibitory antibody, or antigen-binding fragment thereof, in an aqueous solution comprising a buffer system having a pH of 6.0±5%, 20±5% mM histidine, 100±5% mg/mL sucrose, and 0.035±5% polysorbate 80 (w/w). In some embodiments, the MASP-3 inhibitory antibody, or antigen-binding fragment thereof, is included at a concentration of 110 mg/mL±5%. In some embodiments, the MASP-3 inhibitory antibody, or antigen-binding fragment thereof, comprises a heavy chain variable region comprising a HC-CDR1 comprising SEQ ID NO:3; a HC-CDR2 comprising SEQ ID NO:4 or SEQ ID NO:11; and a HC-CDR3 comprising SEQ ID NO:5; and a light chain variable region comprising a LC-CDR1 comprising SEQ ID NO:6 or SEQ ID NO:14; a LC-CDR2 comprising SEQ ID NO:7; and a LC-CDR3 comprising SEQ ID NO:8. In some embodiments, the pharmaceutical composition is sterile. In some embodiments, the MASP-3 inhibitory antibody or antigen-binding fragment thereof comprises a heavy chain variable region comprising at least 80%, 85%, 90%, 95%, 98%, 99% or 100% identical to SEQ ID NO:1, SEQ ID NO:9, or SEQ ID NO:12; and a light chain variable region comprising at least 80%, 85%, 90%, 95%, 98%, 99% or 100% identical to SEQ ID NO:2, SEQ ID NO:10, or SEQ ID NO:13. In some embodiments, the MASP-3 inhibitory antibody, or antigen binding fragment thereof, is selected from the group consisting of a human antibody, a humanized antibody, a chimeric antibody, a murine antibody, and an antigen-binding fragment of any of the foregoing. In some embodiments, the MASP-3 inhibitory antibody or antigen-binding fragment thereof is selected from the group consisting of a single chain antibody, an ScFv, a Fab fragment, an Fab′ fragment, an F(ab′)2 fragment, a univalent antibody lacking a hinge region and a whole antibody. In some embodiments, the MASP-3 inhibitory antibody further comprises an immunoglobulin constant region. In some embodiments, the MASP-3 inhibitory antibody comprises a human IgG4 constant region. In some embodiments, the MASP-3 inhibitory antibody comprises a human IgG4 constant region with an S228P mutation. In some embodiments, the MASP-3 inhibitory antibody comprises a mutation that promotes FcRn interactions at low pH.

The pharmaceutical composition may be present in an article of manufacture containing the pharmaceutical composition comprising a MASP-3 inhibitory antibody, or antigen-binding fragment thereof, in a unit dosage form suitable for therapeutic administration to a human subject, such as a unit dosage in the range of from 10 mg to 1000 mg (such as from 50 mg to 800 mg, or from 75 mg to 500, such as from 100 mg to 300 mg, such as 125 to 275 mg, such as 150 to 200 mg, such as 150±5% mg, 155±5% mg, 160±5% mg, 165±5% mg, 170±5% mg, 175±5% mg, 180±5% mg, 185±5% mg or 190±5% mg) of MASP-3 inhibitory antibody. In some embodiments, the MASP-3 inhibitory antibody, or antigen-binding fragment thereof, comprises a heavy chain variable region comprising a HC-CDR1 comprising SEQ ID NO:3; a HC-CDR2 comprising SEQ ID NO:4 or SEQ ID NO:11; and a HC-CDR3 comprising SEQ ID NO:5; and a light chain variable region comprising a LC-CDR1 comprising SEQ ID NO:6 or SEQ ID NO:14; a LC-CDR2 comprising SEQ ID NO:7; and a LC-CDR3 comprising SEQ ID NO:8.

### V. Methods and Uses

Provided herein are methods for use of an antibody or antigen-binding fragment thereof for treatment of an AP-related disease or disorder. In some embodiments, the method comprises administering to a mammalian subject in need thereof an amount of a MASP-3 antibody or antigen-binding fragment thereof, or composition comprising a MASP-3 antibody or antigen-binding fragment thereof sufficient to inhibit the alternative pathway of complement in the subject. In some embodiments, the subject is a human. In some embodiments, the method can further comprise, prior to administering a compound or composition of this disclosure to the subject, determining that the subject is afflicted with an AP-related disease or disorder. In some embodiments, the AP-related disease or disorder is paroxysmal nocturnal hemoglobinuria (PNH), complement 3 glomerulopathy (C3G), or idiopathic immune complex-mediated glomerulonephritis (ICGN).

In some embodiments, the MASP-3 antibody or antigen-binding fragment has serine protease-inhibitory activity. In some embodiments, the MASP-3 antibody or antigen-binding fragment thereof comprises an antibody referred to in PCT publication WO2018/026722, including the antibodies referred to as 13B1, 10D12, 35C1, 4D5, 1F3, 4B6, and 1A10, along with variants and modified versions of these antibodies thereof. In some embodiments, the MASP-3 antibody or antigen-binding fragment thereof comprises an HCDR1 having the sequence set forth as SEQ ID NO:3, an HCDR2 having the sequence set forth as SEQ ID NO:4 or 11, an HCDR3 having the sequence set forth as SEQ ID NO:5, an LCDR1 having the sequence set forth as SEQ ID NO: 6 or 14, LCDR2 having the sequence set forth as SEQ ID NO:7, and an LCDR3 having the sequence set forth as SEQ ID NO:8.

In some embodiments, the MASP-3 antibody or antigen-binding fragment thereof is administered intravenously. In some embodiments, the MASP-3 antibody or antigen-binding fragment thereof is administered subcutaneously. The MASP-3 antibody or antigen-binding fragment may be administered a single time or may be administered multiple times. When administered multiple times, the timing of administrations may be at pre-set intervals or may be determined on the basis of biomarker measurements or patient status/quality of life. In some embodiments, the MASP-3 antibody or antigen-binding fragment thereof is administered at intervals of 4 to 16 weeks. In some embodiments, the MASP-3 antibody or antigen-binding fragment thereof is administered at intervals of 6 to 12 weeks. In some embodiments, the MASP-3 antibody or antigen-binding fragment thereof is administered at intervals of 6 weeks. In some embodiments, the MASP-3 antibody or antigen-binding fragment thereof is administered at intervals of 8 weeks. In some embodiments, the MASP-3 antibody or antigen-binding fragment thereof is administered at intervals of 10 weeks. In some embodiments, the MASP-3 antibody or antigen-binding fragment thereof is administered at intervals of 12 weeks. In some embodiments, the MASP-3 antibody or antigen-binding fragment thereof is administered at intervals of 14 weeks. In some embodiments, the MASP-3 antibody or antigen-binding fragment thereof is administered at intervals of 16 weeks. The MASP-3 antibody or antigen-binding fragment thereof is administered in an amount sufficient to inhibit the activation of the alternative pathway of complement in the subject. In some embodiments, the MASP-3 antibody or antigen-binding fragment thereof is administered at a dosage in the range from 0.001 mg/kg to 100 mg/kg, such as from 0.05 mg/kg to 50 mg/kg, or from 0.1 mg/kg to 25 mg/kg, or from 0.1 mg/kg to 15 mg/kg, or from 0.1 mg/kg to 10 mg/kg, or from 0.1 mg/kg to 5 mg/kg, or from 0.1 mg/kg to 3 mg/kg, or from 0.1 mg/kg to 1 mg/kg, or from 0.3 mg/kg to 25 mg/kg, or from 0.3 mg/kg to 15 mg/kg, or from 0.3 mg/kg to 10 mg/kg, or from 0.3 mg/kg to 5 mg/kg, or from 0.3 mg/kg to 3 mg/kg, or from 0.3 mg/kg to 1 mg/kg, or from 0.5 mg/kg to 25 mg/kg, or from 0.5 mg/kg to 15 mg/kg, or from 0.5 mg/kg to 10 mg/kg, or from 0.5 mg/kg to 5 mg/kg, or from 0.5 mg/kg to 3 mg/kg, or from 0.5 mg/kg to 1 mg/kg, or from 0.8 mg/kg to 25 mg/kg, or from 0.8 mg/kg to 15 mg/kg, or from 0.8 mg/kg to 10 mg/kg, or from 0.8 mg/kg to 5 mg/kg, or from 0.8 mg/kg to 3 mg/kg, or from 0.8 mg/kg to 1 mg/kg, or from 1 mg/kg to 25 mg/kg, or from 1 mg/kg to 15 mg/kg, or from 1 mg/kg to 10 mg/kg, or from 1 mg/kg to 5 mg/kg, or from 1 mg/kg to 3 mg/kg, or from 3 mg/kg to 25 mg/kg, or from 3 mg/kg to 15 mg/kg, or from 3 mg/kg to 10 mg/kg, or from 3 mg/kg to 5 mg/kg, or from 5 mg/kg to 25 mg/kg, or from 5 mg/kg to 15 mg/kg, or from 5 mg/kg to 10 mg/kg. In some embodiments, the MASP-3 antibody or antigen-binding fragment thereof is administered at a dosage of about 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11, 11.5, 12, 12.5, 13, 13.5 14, 14.5, 15, 15.5, 16, 16.5, 17, 17.5, 18, 18.5, 19, 19.5, or 20 mg/kg. In some embodiments, the MASP-3 antibody or antigen-binding fragment thereof is administered at a dosage of greater than 20 mg/kg. In some embodiments, the MASP-3 antibody or antigen-binding fragment thereof is administered at a dosage of about 1.0 mg/kg. In some embodiments, the MASP-3 antibody or antigen-binding fragment thereof is administered at a dosage of about 3.0 mg/kg. In some embodiments, the MASP-3 antibody or antigen-binding fragment thereof is administered at a dosage of about 5.0 mg/kg. In some embodiments, the MASP-3 antibody or antigen-binding fragment thereof is administered at a dosage of about 10 mg/kg. In some embodiments, the MASP-3 antibody or antigen-binding fragment thereof is administered at a dosage of about 12 mg/kg. In some embodiments, the MASP-3 antibody or antigen-binding fragment thereof is administered at a dosage of about 15 mg/kg. In some embodiments, the MASP-3 antibody or antigen-binding fragment thereof is administered at a dosage of about 17 mg/kg. In some embodiments, the MASP-3 antibody or antigen-binding fragment thereof is administered at a dosage of about 20 mg/kg. In some embodiments, the dosage of MASP-3 antibody or antigen-binding fragment thereof is determined or adjusted on the basis of biomarker measurements of patient status/quality of life.

In some embodiments, the AP-related disease or disorder is paroxysmal nocturnal hemoglobinuria (PNH). In some embodiments, the subject has a hemoglobin level less than 10.5 g/dL. In some embodiments, the subject is being treated or has been treated with a C5 inhibitor, such as ravulizumab or eculizumab. In other embodiments, the subject has not previously been treated with a C5 inhibitor. In some embodiments, the subject has a hemoglobin level less than 10.5 g/dL despite treatment with a C5 inhibitor. The MASP-3 antibody or antigen-binding fragment thereof may be an adjunct therapy or a monotherapy. In some embodiments, the MASP-3 antibody or antigen-binding fragment thereof is an adjunct therapy in conjunction with a C5 inhibitor such as ravulizumab or eculizumab. Treatment with a MASP-3 antibody or antigen-binding fragment thereof may be started as an adjunct therapy and later used as a monotherapy. In some embodiments, the subject is treated with a MASP-3 antibody or antigen-binding fragment thereof in conjunction with a C5 inhibitor such as ravulizumab or eculizumab for a period of time and then, if the subject has shown improvement of PNH symptoms or biomarkers during the adjunct therapy period, the subject is switched to monotherapy with the MASP-3 antibody or antigen-binding fragment thereof. In some embodiments, improvement is identified as an increase in baseline hemoglobin levels. In some embodiments, the subject receives adjunct therapy for 1, 2, 3, 4, 5 6, 7, or 8 doses before switching to monotherapy. A subject may continue treatment with a MASP-3 antibody or antigen-binding fragment thereof, either as adjunctive therapy or monotherapy, for as long as needed to provide continued relief from PNH symptoms. In some embodiments, treatment with a MASP-3 antibody or antigen-binding fragment thereof is continued indefinitely.

Measurements and biomarkers that are relevant to identifying improvement in PNH symptoms and/or efficacy of MASP-3 antibody treatment include hemoglobin levels, indicators of hemolysis (including reticulocytes and lactate dehydrogenase), evidence of ADA, serum concentrations of MASP-3 antibody, serum concentrations of CFD, C3 opsonization of PNH red blood cells (RBCs), PNH RBC clone size, systemic MASP-3 levels, C-reactive protein, D-dimer, number and/or frequency of blood transfusions, and subject scores on the Functional Assessment of Chronic Illness Treatment (FACIT)-Fatigue scale.

In some embodiments, the AP-related disease or disorder is C3 glomerulopathy (C3G) and idiopathic immune complex-mediated glomerulonephritis (ICGN). A subject may continue treatment with a MASP-3 antibody or antigen-binding fragment thereof, either as adjunctive therapy or monotherapy, for as long as needed to provide continued relief from C3G or idiopathic ICGN symptoms. In some embodiments, treatment with a MASP-3 antibody or antigen-binding fragment thereof is continued indefinitely.

Measurements and biomarkers that are relevant to identifying improvement in C3G or idiopathic ICGN symptoms and/or efficacy of MASP-3 antibody treatment include proteinuria levels, serum creatinine levels, glomerular filtration rate, evidence of ADA, serum concentrations of MASP-3 antibody, serum concentrations of CFD, levels of MASP-3, complement factors Bb, C3, and C3a, kidney injury molecule-1 (KIM1), neutrophil gelatinase-associated lipocalin (NGAL), collectin 11, soluble complement complex C5b-9, soluble CD163, MCP-1, and/or clusterin, renal biopsy analysis, and subject scores on the Functional Assessment of Chronic Illness Treatment (FACIT)-Fatigue scale.

Further provided herein is an antibody, antigen-binding fragment, or composition of the present disclosure for use in a method of treating an AP-related disease or disorder. In some embodiments, the use comprises administration to a mammalian subject in need thereof an amount of a MASP-3 antibody or antigen-binding fragment thereof, or composition comprising a MASP-3 antibody or antigen-binding fragment thereof sufficient to inhibit the alternative pathway of complement in the subject. In some embodiments, the subject is a human. In some embodiments, the use can further comprise, prior to administering a compound or composition of this disclosure to the subject, determining that the subject is afflicted with an AP-related disease or disorder. In some embodiments, the MASP-3 antibody or antigen-binding fragment thereof comprises an HCDR1 having the sequence set forth as SEQ ID NO:3, an HCDR2 having the sequence set forth as SEQ ID NO:4 or 11, an HCDR3 having the sequence set forth as SEQ ID NO:5, an LCDR1 having the sequence set forth as SEQ ID NO: 6 or 14, LCDR2 having the sequence set forth as SEQ ID NO:7, and an LCDR3 having the sequence set forth as SEQ ID NO:8. In some embodiments, the AP-related disease or disorder is paroxysmal nocturnal hemoglobinuria (PNH), complement 3 glomerulopathy (C3G), or idiopathic immune complex-mediated glomerulonephritis (ICGN).

Also provided herein is an antibody, antigen-binding fragment, or composition of the present disclosure for use in a method of manufacturing or preparing a medicament for treating an AP-related disease or disorder. In some embodiments, the medicament comprises an amount of a MASP-3 antibody or antigen-binding fragment thereof, or composition comprising a MASP-3 antibody or antigen-binding fragment thereof sufficient to inhibit the alternative pathway of complement in a mammalian subject. In some embodiments, the subject is a human. In some embodiments, the MASP-3 antibody or antigen-binding fragment thereof comprises an HCDR1 having the sequence set forth as SEQ ID NO:3, an HCDR2 having the sequence set forth as SEQ ID NO:4 or 11, an HCDR3 having the sequence set forth as SEQ ID NO:5, an LCDR1 having the sequence set forth as SEQ ID NO: 6 or 14, LCDR2 having the sequence set forth as SEQ ID NO:7, and an LCDR3 having the sequence set forth as SEQ ID NO:8. In some embodiments, the AP-related disease or disorder is paroxysmal nocturnal hemoglobinuria (PNH), complement 3 glomerulopathy (C3G), or idiopathic immune complex-mediated glomerulonephritis (ICGN).

### VI. Sequences

The sequences referred to within the present specification are summarized in TABLE 1.

TABLE 1

| SEQ | | |
|---|---|---|
| ID | | |
| NO. | Description | Sequence |
| 1 | 13B1 VH | QVQLKQSGAELMKPGASVKLSCKATGYTFTGKWIEW |
| | | VKQRPGHGLEWIGEILPGTGSTNYNEKFKGKATFTADS |
| | | SSNTAYMQLSSLTTEDSAMYYCLRSEDVWGTGTTVTV |
| | | SS |
| 2 | 13B1 VL | DIVMTQSPSSLAVSAGEKVTMSCKSSQSLLNSRTRKNY |
| | | LAWYQQKPGQSPKLLIYWASTRESGVPDRFTGSGSGT |
| | | DFTLTISSVQAEDLAVYYCKQSYNIPTFGGGTKLEIKR |
| 3 | 13B1 HCDR1 | GKWIE |
| 4 | 13B1 HCDR2 | EILPGTGSTNYNEKFKG |
| 5 | 13B1 HCDR3 | SEDV |
| 6 | 13B1 LCDR1 | KSSQSLLNSRTRKNYLA |
| 7 | 13B1 LCDR2 | WASTRES |
| 8 | 13B1 LCDR3 | KQSYNIPT |
| 9 | 13B1-9-1 VH | QVQLVQSGAEVKKPGASVKVSCKASGYTFTGKWIEW |
| | | VRQAPGQGLEWIGEILPGTGSTNYAQKFQGRATFTADS |
| | | STSTAYMELSSLRSEDTAVYYCLRSEDVWGQGTLVTV |
| | | SS |
| 10 | 13B1-9-1 VL | DIVMTQSPDSLAVSLGERATINCKSSQSLLNSRTRKNY |
| | | LAWYQQKPGQPPKLLIYWASTRESGVPDRESGSGSGT |
| | | DFTLTISSLQAEDVAVYYCKQSYNIPTFGQGTKVEIKR |
| 11 | 13B1-9-1 HCDR2 | EILPGTGSTNYAQKFQG |
| 12 | 13B1-10-1 VH | QVQLVQSGAEVKKPGASVKVSCKASGYTFTGKWIEW |
| | | VRQAPGQGLEWIGEILPGTGSTNYNEKFKGRATFTADS |
| | | STSTAYMELSSLRSEDTAVYYCLRSEDVWGQGTLVTV |
| | | SS |
| 13 | 13B1-9-1-NA VL | DIVMTQSPDSLAVSLGERATINCKSSQSLLASRTRKNY |
| | | LAWYQQKPGQPPKLLIYWASTRESGVPDRFSGSGSGT |
| | | DFTLTISSLQAEDVAVYYCKQSYNIPTFGQGTKVEIKR |
| 14 | 13B1-9-1-NA | KSSQSLLASRTRKNYLA |
| | LCDR1 | |
| 15 | 13B1-10-1-NA | DIVMTQSPDSLAVSLGERATINCKSSQSLLASRTRKNY |
| | Light Chain | LAWYQQKPGQPPKLLIYWASTRESGVPDRESGSGSGT |
| | | DFTLTISSLQAEDVAVYYCKQSYNIPTFGQGTKVEIKRT |
| | | VAAPSVFIFPPSDEQLKSGTASVVCLLNNFYPREAKVQ |
| | | WKVDNALQSGNSQESVTEQDSKDSTYSLSSTLTLSKA |
| | | DYEKHKVYACEVTHQGLSSPVTKSFNRGEC |
| 16 | 13B1-10-1-NA | QVQLVQSGAEVKKPGASVKVSCKASGYTFTGKWIEW |
| | Heavy Chain | VRQAPGQGLEWIGEILPGTGSTNYNEKFKGRATFTADS |
| | | STSTAYMELSSLRSEDTAVYYCLRSEDVWGQGTLVTV |
| | | SSASTKGPSVFPLAPCSRSTSESTAALGCLVKDYFPEPV |
| | | TVSWNSGALTSGVHTFPAVLQSSGLYSLSSVVTVPSSS |
| | | LGTKTYTCNVDHKPSNTKVDKRVESKYGPPCPPCPAPE |
| | | FLGGPSVFLFPPKPKDTLMISRTPEVTCVVVDVSQEDPE |
| | | VQFNWYVDGVEVHNAKTKPREEQFNSTYRVVSVLTV |
| | | LHQDWLNGKEYKCKVSNKGLPSSIEKTISKAKGQPREP |
| | | QVYTLPPSQEEMTKNQVSLTCLVKGFYPSDIAVEWESN |
| | | GQPENNYKTTPPVLDSDGSFFLYSRLTVDKSRWQEGN |
| | | VFSCSVLHEALHSHYTQKSLSLSLGK |
| 17 | Human MASP-3 | MRWLLLYYALCFSLSKASAHTVELNNMFGQIQSPGYP |
| | (with 19 aa leader) | DSYPSDSEVTWNITVPDGFRIKLYFMHFNLESSYLCEY |
| | | DYVKVETEDQVLATFCGRETTDTEQTPGQEVVLSPGSF |
| | | MSITFRSDESNEERFTGFDAHYMAVDVDECKEREDEEL |
| | | SCDHYCHNYIGGYYCSCRFGYILHTDNRTCRVECSDNL |
| | | FTQRTGVITSPDFPNPYPKSSECLYTIELEEGFMVNLQF |
| | | EDIFDIEDHPEVPCPYDYIKIKVGPKVLGPFCGEKAPEPI |
| | | STQSHSVLILFHSDNSGENRGWRLSYRAAGNECPELQP |
| | | PVHGKIEPSQAKYFFKDQVLVSCDTGYKVLKDNVEMD |
| | | TFQIECLKDGTWSNKIPTCKIVDCRAPGELEHGLITFST |
| | | RNNLTTYKSEIKYSCQEPYYKMLNNNTGIYTCSAQGV |
| | | WMNKVLGRSLPTCLPECGQPSRSLPSLVKRIIGGRNAE |
| | | PGLFPWQALIVVEDTSRVPNDKWFGSGALLSASWILTA |
| | | AHVLRSQRRDTTVIPVSKEHVTVYLGLHDVRDKSGAV |
| | | NSSAARVVLHPDFNIQNYNHDIALVQLQEPVPLGPHV |
| | | MPVCLPRLEPEGPAPHMLGLVAGWGISNPNVTVDEIIS |
| | | SGTRTLSDVLQYVKLPVVPHAECKTSYESRSGNYSVTE |
| | | NMFCAGYYEGGKDTCLGDSGGAFVIFDDLSQRWVVQ |
| | | GLVSWGGPEECGSKQVYGVYTKVSNYVDWVWEQMG |
| | | LPQSVVEPQVER |
| 18 | Linker | GGGGS |
| 19 | Linker | GGGGSGGGGSGGGG |

### VII. Examples

### Example 1

### Phase 1 Single Ascending Dose Study in Healthy Subjects

A Phase 1 clinical trial was conducted to evaluate the safety, tolerability, pharmacokinetics (PK), and pharmacodynamics (PD) of antibody 13B1-10-1-NA in healthy human subjects. The Phase 1 study was randomized, double-blind, and placebo-controlled, and was carried out in a single center.

Subjects were healthy males and females aged 18 to 64 years at screening and had a body mass index (BMI) of 20-32 kg/m² and a weight of at least 50 kg. Of the 72 subjects in the study, 37 were female and 35 were male. The median age was 42 years, with a range from 20-63 years. The median BMA was 27.2 kg/m², with a range from 21.0 to 31.4 kg/m². The median weight was 77.0 kg, with a range from 50.8 to 105.7 kg. The subjects' racial breakdown was as follows: 40 white, 22 black or African American, 3 Asian, 2 American Indian or Alaska Native, and 5 reporting multiple races. Of the 72 subjects, 4 reported Hispanic or Latino ethnicity.

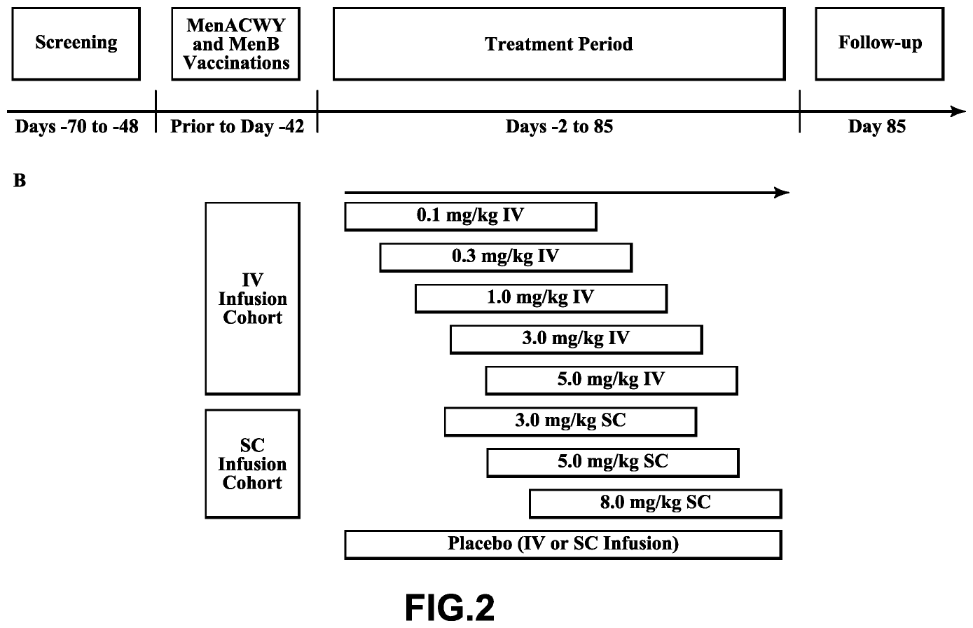

Antibody 13B1-10-1-NA was administered intravenously (IV) at 0.1 mg/kg, 0.3 mg/kg, 1.0 mg/kg, 3.0 mg/kg, or 5.0 mg/kg or subcutaneously (SC) at 3.0 mg/kg, 5.0 mg/kg, or 8.0 mg/kg, or the subject was administered an IV or SC placebo. A graphic representation of the study design is shown in FIG. 2.

The study measured serum 13B1-10-1-NA concentrations, serum PK parameters (Cₘₐₓ, Tₘₐₓ, t_{1/2}, ACU_{0-inf}, CL, CL/F, V_{z}, V₅₅, V_{z}/F), change from baseline in mature factor D plasma concentrations, incidence of anti-drug antibodies in serum, and occurrence of adverse events. Pharmacokinetic results for subjects administered 13B1-10-1-NA by IV (30 subjects) or by SC (24 subjects) are provided in TABLE 2.

TABLE 2

| Parameter | IV Administration | | SC Administration | |
|---|---|---|---|---|
| Serum concentration: | | | | |
| Geometric mean Cₘₐₓ | 3.2-1.39.0 | μg/mL | 134.0-388.0 | μg/mL |
| Geometric mean | 0.3-53.2 | h · mg/mL | 1.7-9.5 | h · mg/mL |
| ACU_{0-inf} | | | | |
| Median Tₘₐₓ | 0.7-2.5 | h | 96-239 | h |
| Geometric mean t_{1/2} | 94-399 | h | 239-406 | h |

| Geometric mean | | | |
|---|---|---|---|
| clearance: | | | |
| CL | 7.7-32.1 | | — |
| CL/F | — | | 14.1-28.3 |
| Geometric mean | | | |
| volume of | | | |
| distribution: | | | |
| V₅₅ | 3.0-5.4 | L | — |
| V_{z} | 4.0-5.5 | L | |

| | | |
|---|---|---|
| V_{z}/F | — | 6.7-12.4 |

The PK properties were observed to be dose-proportional (with non-linearity) for both IV and SC administration. A long half-life (geometric mean range 94-406 hours) was observed, with measurable drug concentrations detected at day 85 for cohorts receiving either IV administration (3 mg/kg or 5 mg/kg) and SC administration (3 mg/kg, 5 mg/kg, or 8 mg/kg).

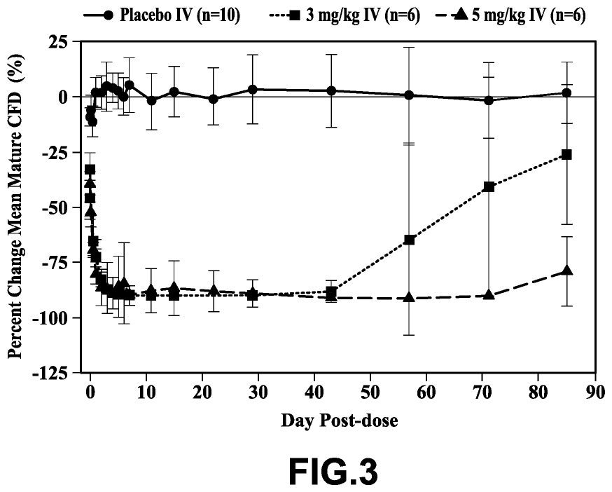

Pharmacodynamic results are shown in FIG. 3. The percent change in mean mature complement factor D (CFD), the key PD marker for AP activity, showed a dose-proportional response with rapid suppression of mature CFD levels. A substantial degree of suppression was observed over a long duration in subjects who received 3 or 5 mg/kg IV administration, as compared to subjects who received placebo. The lower limit of quantification in this assay was 43.9 ng/mL. Values measured below this threshold were assigned a value of 43.9 ng/mL.

Antibody 13B1-10-1-NA was well tolerated. Most observed treatment-emergent adverse events (TEAEs) were mild and short in duration. A summary of adverse events (AEs) is provided in TABLE 3 for subjects administered 13B1-10-1-NA by IV (40 subjects) or by SC (32 subjects).

TABLE 3

| | IV Administration | | SC administration | | |
|---|---|---|---|---|---|
| | Antibody | Placebo | Antibody | Placebo | |
| | (30 subjects) | (10 subjects) | (24 subjects) | (8 subjects) | Overall |
| AE, number of | 11 | 5 | 20 | 3 | 39 |
| subjects affected | | | | | |
| AE, number of events | 8 | 14 | 42 | 11 | 85 |
| AE, mild events | 8 | 4 | 13 | 2 | 27 |
| AE, moderate events | 2 | 1 | 2 | 1 | 6 |
| AE, severe events | 1 | 0 | 0 | 0 | 1 |
| Injection site reaction | 1 | | 17 | 2 | 20 |
| (ISR), number of | | | | | |
| subjects affected | | | | | |
| ISR, number of events | 1 | | 27 | 3 | 31 |
| ISR, bruising | 0 | 0 | 3 | 0 | 3 |
| ISR, discomfort | 0 | 0 | 0 | 1 | 1 |
| ISR, erythema | 0 | 0 | 8 | 1 | 9 |
| ISR, induration | 0 | 0 | 14 | 1 | 15 |
| ISR, warmth | 0 | 0 | 1 | 0 | 1 |
| ISR, swelling | 0 | 0 | 1 | 0 | 1 |
| AE leading to study | 0 | 0 | 0 | 0 | 0 |
| discontinuation | | | | | |
| TEAEs, number of | 2 | 0 | 16 | 2 | 20 |
| subjects affected | | | | | |
| TEAEs, number of | 3 | 0 | 26 | 3 | 32 |
| events | | | | | |

The overall confirmed positive rate of ADAs was 14.8% in subjects receiving antibody 13B1-10-1-NA. There were no incidences of hypersensitivity reaction or of anaphylaxis. There was no evidence of an impact on PK or PD from ADAs.

### Example 2

### Phase 1b Study in PNH Patients with Sub-Optimal Response to Ravulizumab

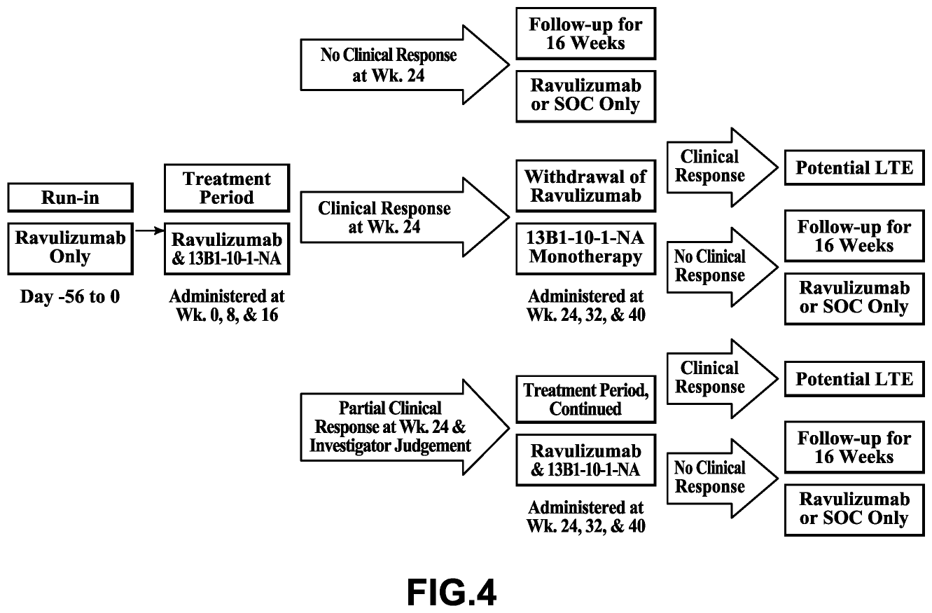

A Phase 1b study is conducted to assess safety and tolerability, along with PK, PD, and certain measurements of efficacy, in PNH patients with a sub-optimal response to ravulizumab treatment. This study is a multicenter, open-label, uncontrolled study. Subjects are PNH patients having a hemoglobin level less than 10.5 g/dL when treated with ravulizumab. A graphical representation of such a study is provided in FIG. 4.

Subjects are males or females of at least 18 years of age with PNH who are on stable treatment with ravulizumab administered every 8 weeks by IV infusion, and who have a sub-optimal response to this treatment, defined as a hemoglobin level of less than 10.5 g/dL despite ravulizumab treatment. Up to 12 total subjects are expected to be enrolled, with 4 to 6 patients per dosing cohort.

Antibody 13B1-10-1-NA is evaluated as adjunctive therapy in addition to ravulizumab and as monotherapy. For at least 8 weeks prior to the study and for an 8-week run-in period, subjects receive only ravulizumab on their regular schedule of one dose every 8 weeks. After the 8-week run-in period, subjects are administered 3 IV doses of ravulizumab and 3 IV doses of antibody 13B1-10-1-NA. The ravulizumab and 13B1-10-1-NA doses are administered on the same day at weeks 0, 8, and 16 of the study. One cohort receives antibody 13B1-10-1-NA at 3 mg/kg and a second cohort receives antibody 13B1-10-1-NA at 5 mg/kg. Samples for PK, PD, ADA, and biomarker analysis are taken before each dosing and at specified intervals during the follow-up period. An independent Data and Safety Monitoring Committee (DSMC) reviews safety and tolerability data after the first subject has completed 3 doses in each cohort and after 3 subjects have completed 3 doses each in each cohort. The DSMC continues to review safety and tolerability data at intervals throughout the study. Subjects who show an incomplete response at week 24, defined as a partial increase from the individual subject's baseline hemoglobin level, may continue with adjunctive treatment for an additional 3 doses, to be administered at weeks 24, 32, and 40.

Subjects who demonstrate an increase in hemoglobin levels of at least 2.0 g/dL from baseline at week 24 are discontinued from treatment with ravulizumab and continued with 13B1-10-1-NA monotherapy. Dosing with 13B1-10-1-NA is continued at 8 week intervals at the dose designated for the subject's cohort and the response to monotherapy is assessed at 4 week intervals. Subjects continue with 13B1-10-1-NA monotherapy unless hemoglobin levels fall below the subject's baseline and/or their clinical condition warrants discontinuation. Subjects who continue to show sustained clinical response at week 40 are eligible to continue 13B1-10-1-NA treatment as part of a long-term extension study. Subjects discontinued from 13B1-10-1-NA monotherapy are returned to treatment with ravulizumab or other standard of care (SOC) therapies. Subjects that experience breakthrough hemolysis are treated with any approved C5 inhibitor, including ravulizumab or eculizumab and/or blood transfusion, as per SOC.

Subjects who show no clinical response at week 24, defined as an increase from the subject's baseline hemoglobin level, are returned to treatment with ravulizumab only or other SOC therapy.

Subjects are monitored for a 16 week follow-up period at the conclusion of the adjunctive therapy phase (weeks 0-24 or weeks 0-40) or at the conclusion of the monotherapy phase for those subjects that enter monotherapy (duration of monotherapy dependent on clinical response).

Samples taken during the study are analyzed for a variety of components, including hemoglobin levels, indicators of hemolysis (including reticulocytes and lactate dehydrogenase), evidence of ADA, serum concentrations of antibody 13B1-10-1-NA, serum concentrations of CFD and other serum/plasma PD parameters, and a variety of biomarkers such as C3 opsonization of PNH red blood cells (RBCs), PNH RBC clone size, systemic MASP-3 levels, C-reactive protein, D-dimer, etc. Subjects are also monitored for number of blood transfusions during the study and quality of life, which is assessed using the Functional Assessment of Chronic Illness Treatment (FACIT)-Fatigue scale.

### Example 3

### Phase 1b Study in PNH Patients, Including C5-Inhibitor-Treated and/or C5-Inhibitor-Untreated Patients

A Phase 1b study is conducted to assess safety and tolerability, along with PK, PD, and certain measurements of efficacy, of antibody 13B1-10-1-NA in PNH patients. This study is a multicenter, open-label, uncontrolled study. Subjects are PNH patients who are showing an inadequate response to eculizumab or ravulizumab treatment or who are not currently or have not previously received complement inhibitor treatment. Inadequate response is defined as having a hemoglobin level less than 10.5 g/dL when treated with eculizumab or ravulizumab.

Subjects are males or females of at least 18 years of age with PNH who are on stable treatment with eculizumab or ravulizumab for at least 6 months at the time of screening and have an inadequate response to treatment, or who are not receiving complement inhibitor treatment at the time of screening. Up to approximately 10 total subjects are enrolled.

Subjects receive subcutaneous (SC) dosing of antibody 13B1-10-1-NA every four weeks for a total of 13 doses. Antibody 13B1-10-1-NA is administered at 5 mg/kg. Subjects completing the 48 week treatment period may be eligible for a long-term extension study. Occurrence of breakthrough hemolysis in a subject is treated with ravulizumab or eculizumab and/or blood transfusion, in accordance with SOC. After the treatment period, subjects are monitored for an 8-week follow-up period.

Samples for PK, PD, ADA, and biomarker analysis are taken before each dosing and at specified intervals during the follow-up period. Samples are analyzed for a variety of components, including hemoglobin levels, bilirubin levels, indicators of hemolysis (including reticulocytes and lactate dehydrogenase (LDH)), evidence of ADA, serum concentrations of antibody 13B1-10-1-NA, serum concentrations of CFD and other serum/plasma PD parameters, and a variety of biomarkers such as C3 opsonization of PNH red blood cells (RBCs), PNH RBC clone size, systemic MASP-3 levels, etc. Subjects are also monitored for number of blood transfusions during the study.

Interim results showed statistically significant and clinically meaningful improvements in all measured marks of hemolysis. A first set of interim results were obtained from eight complement-inhibitor-naïve adults with PNH who were treated with antibody 13B1-10-1-NA at a dose of 5 mg/kg administered SC every four weeks, as described above. Interim results for eight subjects through time points up to day 85 after first dosing are shown in Table 4. P values shown are for change from zero using t-test.

TABLE 4

| | Baseline | Day 8 | Day 15 | Day 29 | Day 57 | Day 85 |
|---|---|---|---|---|---|---|
| Number of subjects | 8 | 8 | 7 | 7 | 3 | 3 |

LDH

| | | | | | | |
|---|---|---|---|---|---|---|
| Mean | 2046.6 | 652.24 | 343.26 | 382.11 | 718.77 | 342.13 |
| Change from baseline | — | −1394.34 | −1498.54 | −1459.69 | −1532.23 | −1908.87 |
| P-value | — | <0.001 | <0.001 | 0.001 | 0.064 | 0.003 |

Total Bilirubin

| | | | | | | |
|---|---|---|---|---|---|---|
| Mean | 36.08 | 19.41 | 13.91 | 15.75 | 36.90 | 20.6 |
| Change from baseline | — | −16.66 | −20.40 | −18.56 | −0.47 | −16.77 |
| P-value | — | 0.067 | <0.001 | 0.001 | 0.980 | 0.055 |

Hemoglobin

| | | | | | | |
|---|---|---|---|---|---|---|
| Mean | 6.34 | 7.21 | 7.99 | 9.30 | 10.67 | 12.4 |
| Change from baseline | — | 0.88 | 1.89 | 3.20 | 4.53 | 6.27 |
| P-value | — | 0.003 | 0.004 | 0.008 | 0.004 | 0.005 |

Abs Reticulocytes

| | | | | | | |
|---|---|---|---|---|---|---|
| Mean | 0.191 | 0.096 | 0.085 | 0.058 | 0.168 | 0.084 |
| Change from baseline | — | −0.095 | −0.090 | −0.117 | −0.022 | −0.105 |
| P-value | — | 0.011 | 0.020 | 0.002 | 0.851 | 0.011 |

The baseline mean hemoglobin (Hgb) was 6.34 g/dL. By Day 57 (after 2 doses), all treated subjects achieved an increase in Hgb of 4.0 g/dL or more. By Day 85 (following 3 doses), the mean Hgb was 12.4 g/dL with a mean change from baseline of 6.27 g/dL (p=0.005). Improvement was rapid, with significant mean Hgb improvement of 0.88 g/dL (p=0.003) seen at the first timepoint (Day 8), persistently increasing and remaining statistically significant through the last observed timepoint (Day 85). The mean baseline LDH was 2067, more than 8 times the upper limit of normal. Statistically significant improvements in LDH were observed at Day 8, the first measured timepoint, with subsequent and substantial further reductions observed during the study period.

13B1-10-1-NA was observed to be safe and well tolerated. No treated subjects required or received blood transfusions following treatment initiation.

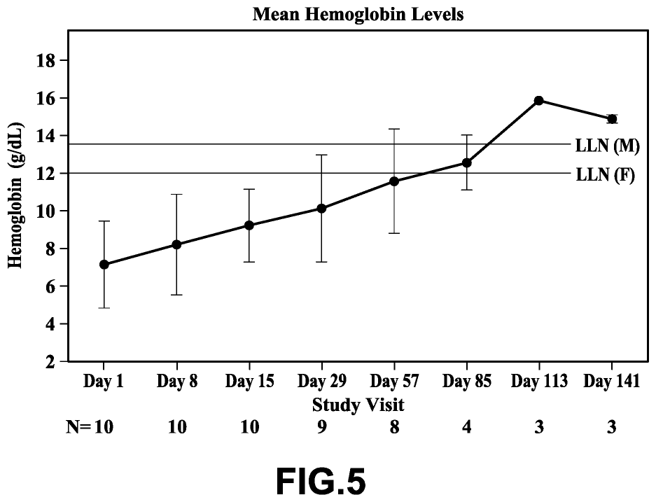

A second set of interim results were obtained from ten complement-inhibitor-naïve adults with PNH who were treated with antibody 13B1-10-1-NA at a dose of 5 mg/kg administered SC every four weeks, as described above. Interim results for ten subjects through time points up to day 141 after first dosing are shown in FIGS. 5-11. The patients were adults with confirmed PNH diagnosis by flow cytometry (clone size >10%), were complement inhibitor treatment-naïve, had starting hemoglobin levels of <10.5 g/kL, and starting LDH levels greater than 1.5 time the upper limit of normal. At the time of this interim data collection, ten patients had received one or more doses of antibody 13B1-10-1-NA, eight patients had received two or more doses, four patients had received three or more doses, and three patients had received five doses. Seven of the ten patients had received RBC transfusions in the twelve months prior to the first dose. None of the ten patients required transfusions during the study period.

FIG. 5 shows the mean hemoglobin levels in the ten patients over time after first dosing, through day 141. The number of patients contributing to the data at each time point is indicated below the x-axis. The horizontal lines indicate the lower limit of normal for males (LLN(M)) and for females (LLN(F)), as labeled. After the first dose, mean hemoglobin levels were up 3.3 g/dL from baseline, p=0.001. After five doses, mean hemoglobin levels were up 8.7 g/dL from baseline, p=0.018.

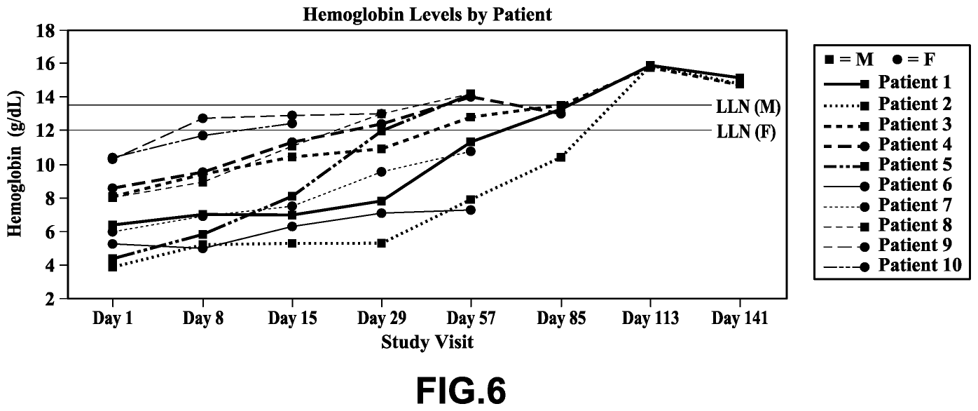

FIG. 6 shows the hemoglobin levels in each of the ten patients over time after first dosing. The horizontal lines indicate lower limit of normal for males (LLN(M)) and lower limit of normal for females (LLN(F)), as labeled. Male patients are indicated with squares; female patients are indicated with circles. All ten patients had an increase in hemoglobin greater than or equal to 2 g/dL and eight of the ten patients had an increase in hemoglobin greater than or equal to 12 g/kL. The two patients who showed a lower increase in hemoglobin (patients 6 and 7) also suffered from myelodysplastic syndrome (MDS).

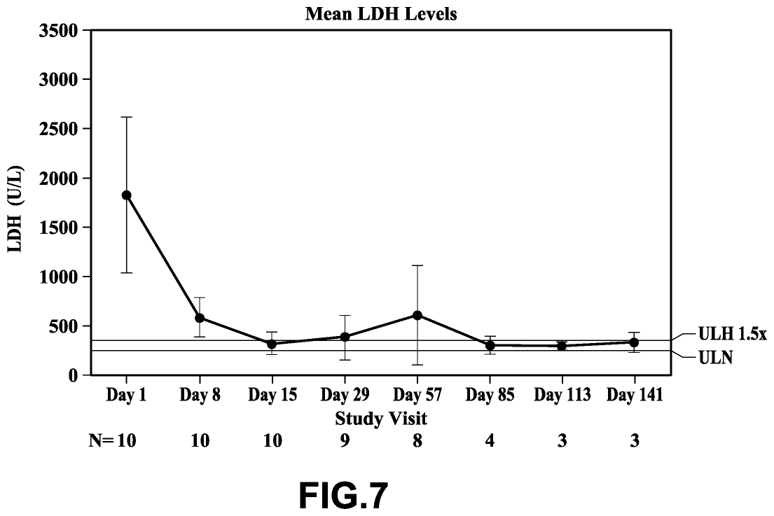

FIG. 7 shows the mean LDH levels in the ten patients over time after first dosing, through day 141. The number of patients contributing to the data at each time point is indicated below the x-axis. The horizontal lines indicate the upper limit of normal (ULN) and 1.5× the upper limit of normal (ULN 1.5×), as labeled. After the first dose, mean LDH levels were down 1548 U/L from baseline, p=0.001. After five doses, mean LDH levels were down 1916 U/L from baseline, p=0.003.

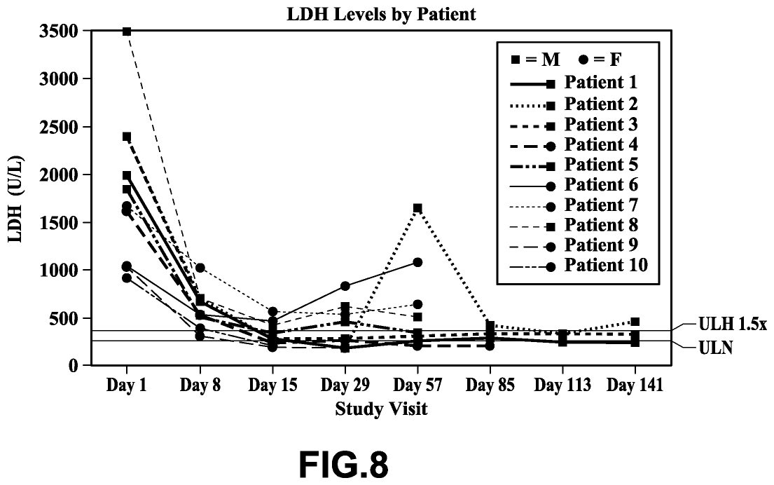

FIG. 8 shows the LDL levels in each of the ten patients over time after first dosing. The horizontal lines indicate upper limit of normal (ULN) and 1.5× the upper limit of normal (ULN 1.5×), as labeled. Three patients had increases in LDH at or near the end of a dosing period, although hemoglobin levels were not reduced in any of these patients. This information will help inform future dosing levels and frequencies.

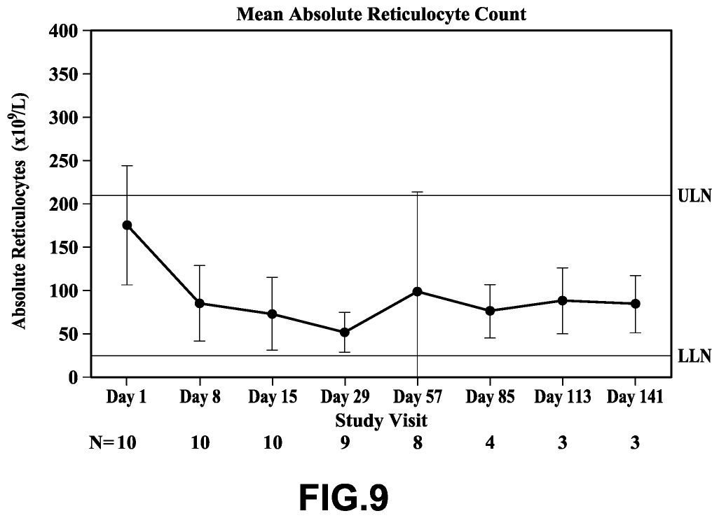

FIG. 9 shows the mean absolute reticulocyte count in the ten patients over time after first dosing, through day 141. The number of patients contributing to the data at each time point is indicated below the x-axis. The horizontal lines indicate the upper limit of normal (ULN) and the lower limit of normal (LLN), as labeled. After the first dose, mean absolute reticulocyte counts were reduced by 130×10⁹/L from baseline, p=0.001. After five doses, mean absolute reticulocyte counts were down by 106×10⁹/L from baseline, p=0.015. Mean reticulocyte counts were reduced from baseline by 90-133×10⁹/L at all timepoints.

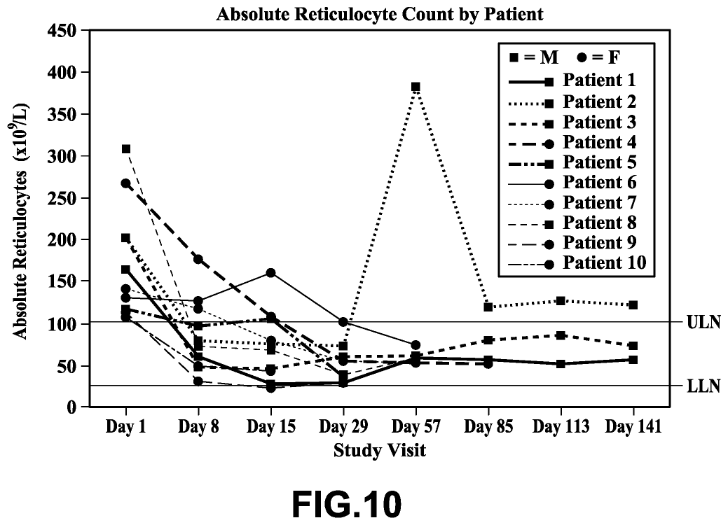

FIG. 10 shows the absolute reticulocyte count in each of the ten patients over time after first dosing. The horizontal lines indicate upper limit of normal (ULN) and lower limit of normal (LLN), as labeled.

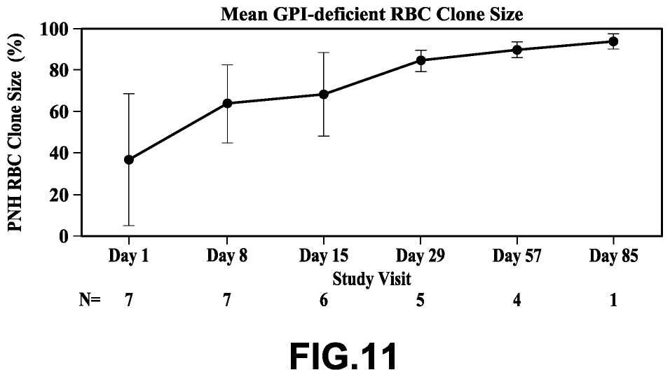

FIG. 11 shows the mean GPI-deficient (glycosylphosphatidylinositol-deficient) RBC clone size in seven patients over time after first dosing, through day 85. The number of patients contributing to the data at each time point is indicated below the x-axis. After two doses, the mean RBC clone size was up 38.4% from baseline, p=0.077.

As shown by these data, antibody 13B1-10-1-NA provided normalization of hemoglobin levels in eight of ten patients with monthly SC dosing without clinical breakthrough hemolysis. Antibody 13B1-10-1-NA also provided normalization of LDH in seven of ten patients, normalization of reticulocytes in nine of ten patients, and achieved transfusion independence for all ten patients.

Based on this data, and on pharmacokinetic data in both healthy volunteers and patients with PNH, it is expected that once quarterly may be an effective dosing frequency for antibody 13B1-10-1-NA when administered either SC or IV.

### Example 4

### Phase 1b Study in C3G and Idiopathic ICGN Patients

A Phase 1b study is conducted to assess safety and tolerability, along with PK, PD, and certain measurements of efficacy, of antibody 13B1-10-1-NA in C3 glomerulopathy (C3G) and idiopathic immune complex-mediated glomerulonephritis (ICGN) patients. This study is a multicenter, open-label, uncontrolled study. Subjects are C3G patients or idiopathic ICGN patients, with diagnosis confirmed by biopsy within 36 months of screening.

Subjects are males or females of at least 18 years of age with C3G or idiopathic ICGN who are on stable treatment with angiotensin converting enzyme (ACE) inhibitors or angiotensin receptor blockers (ARB) for at least 90 days at the time of screening. Up to approximately 10 C3G patients and up to approximately 10 ICGN patients are enrolled.

Subjects receive subcutaneous (SC) dosing of antibody 13B1-10-1-NA every four weeks for a total of 13 doses. Antibody 13B1-10-1-NA is administered at 5 mg/kg. Subjects completing the 48 week treatment period may be eligible for a long-term extension study. After the treatment period, subjects are monitored for an 8-week follow-up period.

Samples for PK, PD, ADA, and biomarker analysis are taken before each dosing and at specified intervals during the follow-up period. Samples are analyzed for a variety of components, including proteinuria levels, creatinine, evidence of ADA, serum concentrations of antibody 13B1-10-1-NA, serum concentrations of CFD and other serum/plasma PD parameters. Subjects may elect to participate in a renal biopsy, which is used to identify changes from baseline in renal histopathology at week 24.

### VIII. Other Embodiments

All publications, patent applications, and patents mentioned in this specification are herein incorporated by reference.

While certain embodiments of the invention have been illustrated and described, it will be appreciated that various changes can be made therein without departing from the spirit and scope of the invention. Although the invention has been described in connection with specific embodiments, it should be understood that the invention as claimed should not be unduly limited to such specific embodiments. Indeed, various modifications of the specific embodiments described that are obvious to those skilled in the fields of medicine, immunology, pharmacology, or related fields are intended to be within the scope of the invention.

Accordingly, the following numbered paragraphs describing specific embodiments are provided for clarity, but should not be construed to limit the claims.

1. A method for treating a human subject suffering from paroxysmal nocturnal hemoglobinuria (PNH), the method comprising administering to the subject an amount of a MASP-3 inhibitory agent sufficient to inhibit alternative pathway complement activation.

2. The method of paragraph 1, wherein the subject exhibits a sub-optimal response to treatment with a C5 inhibitor.

3. The method of paragraph 2, wherein the C5 inhibitor is eculizumab, ravulizumab, or a biosimilar to either eculizumab or ravulizumab.

4. The method of paragraph 2, wherein the subject exhibits a hemoglobin level of less than 10.5 g/dL in response to C5 inhibitor treatment.

5. The method of any of paragraphs 1-4, wherein the MASP-3 inhibitory agent is an anti-MASP-3 antibody or antigen-binding fragment thereof.

6. The method of paragraph 5, wherein the anti-MASP-3 antibody or antigen-binding fragment thereof comprises a heavy chain variable region comprising an HCDR1 having the sequence set forth as SEQ ID NO:3, an HCDR2 having the sequence set forth as SEQ ID NO:4 or 11, and an HCDR3 having the sequence set forth as SEQ ID NO:5 and a light chain variable region comprising an LCDR1 having the sequence set forth as SEQ ID NO: 6 or 14, an LCDR2 having the sequence set forth as SEQ ID NO:7, and an LCDR3 having the sequence set forth as SEQ ID NO:8.

7. The method of paragraph 6, wherein the anti-MASP-3 antibody or antigen-binding fragment thereof comprises an HCDR1 having the sequence set forth as SEQ ID NO:3, an HCDR2 having the sequence set forth as SEQ ID NO:4, an HCDR3 having the sequence set forth as SEQ ID NO:5, an LCDR1 having the sequence set forth as SEQ ID NO:6, an LCDR2 having the sequence set forth as SEQ ID NO:7, and an LCDR3 having the sequence set forth as SEQ ID NO:8.

8. The method of paragraph 7, wherein the anti-MASP-3 antibody or antigen-binding fragment thereof comprises a VH having the sequence set forth as SEQ ID NO:1 and a VL having the sequence set forth as SEQ ID NO:2.

9. The method of paragraph 7, wherein the anti-MASP-3 antibody or antigen-binding fragment thereof comprises a VH having the sequence set forth as SEQ ID NO:12 and a VL having the sequence set forth as SEQ ID NO:10.

10. The method of paragraph 6, wherein the anti-MASP-3 antibody or antigen-binding fragment thereof comprises an HCDR1 having the sequence set forth as SEQ ID NO:3, an HCDR2 having the sequence set forth as SEQ ID NO: 11, an HCDR3 having the sequence set forth as SEQ ID NO:5, an LCDR1 having the sequence set forth as SEQ ID NO:6, an LCDR2 having the sequence set forth as SEQ ID NO:7, and an LCDR3 having the sequence set forth as SEQ ID NO:8.

11. The method of paragraph 10, wherein the anti-MASP-3 antibody or antigen-binding fragment thereof comprises a VH having the sequence set forth as SEQ ID NO:9 and a VL having the sequence set forth as SEQ ID NO:10.

12. The method of paragraph 6, wherein the anti-MASP-3 antibody or antigen-binding fragment thereof comprises an HCDR1 having the sequence set forth as SEQ ID NO:3, an HCDR2 having the sequence set forth as SEQ ID NO:11, and an HCDR3 having the sequence set forth as SEQ ID NO:5, an LCDR1 having the sequence set forth as SEQ ID NO:14, an LCDR2 having the sequence set forth as SEQ ID NO:7, and an LCDR3 having the sequence set forth as SEQ ID NO:8.

13. The method of paragraph 12, wherein the anti-MASP-3 antibody or antigen-binding fragment thereof comprises a VH having the sequence set forth as SEQ ID NO:9 and a VL having the sequence set forth as SEQ ID NO:13.

14. The method of paragraph 6, wherein the anti-MASP-3 antibody or antigen-binding fragment thereof comprises an HCDR1 having the sequence set forth as SEQ ID NO:3, an HCDR2 having the sequence set forth as SEQ ID NO:4, an HCDR3 having the sequence set forth as SEQ ID NO:5, an LCDR1 having the sequence set forth as SEQ ID NO:14, an LCDR2 having the sequence set forth as SEQ ID NO:7, and an LCDR3 having the sequence set forth as SEQ ID NO:8.

15. The method of paragraph 14, wherein the anti-MASP-3 antibody or antigen-binding fragment thereof comprises a VH having the sequence set forth as SEQ ID NO:12 and a VL having the sequence set forth as SEQ ID NO:13.

16. The method of paragraph 15, wherein the anti-MASP-3 antibody or antigen-binding fragment thereof comprises a light chain having the sequence set forth as SEQ ID NO:15 and a heavy chain having the sequence set forth as SEQ ID NO:16

17. The method of any one of paragraphs 1-16, wherein the MASP-3 inhibitory agent is administered subcutaneously or intravenously.

18. The method of any one of paragraphs 1-17, wherein the MASP-3 inhibitory agent is administered at intervals of 4 to 16 weeks.

19. The method of paragraph 18, wherein the MASP-3 inhibitory agent is administered at intervals of 6 to 12 weeks.

20. The method of paragraph 18, wherein the MASP-3 inhibitory agent is administered at intervals of 4 weeks.

21. The method of paragraph 18, wherein the MASP-3 inhibitory agent is administered at intervals of 8 weeks.

22. The method of paragraph 18, wherein the MASP-3 inhibitory agent is administered at intervals of 12 weeks.

23. The method of any one of paragraphs 1-22, wherein the MASP-3 inhibitory agent is administered at a dosage of 0.1 mg/kg to 50 mg/kg.

24. The method of paragraph 23, wherein the MASP-3 inhibitory agent is administered at a dosage of 1 mg/kg to 25 mg/kg.

25. The method of paragraph 23, wherein the MASP-3 inhibitory agent is administered at a dosage of 1.0 mg/kg to 15.0 mg/kg.

26. The method of paragraph 23, wherein the MASP-3 inhibitory agent is administered at a dosage of about 1.0 mg/kg.

27. The method of paragraph 23, wherein the MASP-3 inhibitory agent is administered at a dosage of about 3.0 mg/kg.

28. The method of paragraph 23, wherein the MASP-3 inhibitory agent is administered at a dosage of about 5.0 mg/kg.

29. The method of paragraph 23, wherein the MASP-3 inhibitory agent is administered at a dosage of about 7.0 mg/kg.

30. The method of paragraph 23, wherein the MASP-3 inhibitory agent is administered at a dosage of about 10 mg/kg.

31. The method of paragraph 23, wherein the MASP-3 inhibitory agent is administered at a dosage of about 12 mg/kg.

32. The method of paragraph 23, wherein the MASP-3 inhibitory agent is administered at a dosage of about 15 mg/kg.

33. The method of paragraph 23, wherein the MASP-3 inhibitory agent is administered at a dosage of about 17 mg/kg.

34. The method of paragraph 23, wherein the MASP-3 inhibitory agent is administered at a dosage of about 20 mg/kg.

35. The method of any one of paragraphs 23-34, wherein a pharmaceutical composition is administered to the subject, the pharmaceutical composition comprising a MASP-3 inhibitory antibody, or antigen-binding fragment thereof, in an aqueous solution.

36. The method of paragraph 35, wherein the pharmaceutical composition comprises a MASP-3 inhibitory antibody, or antigen-binding fragment thereof, in an aqueous solution comprising a buffer system having a pH of 6.0±5%, 20±5% mM histidine, 100±5% mg/mL sucrose, and 0.035±5% polysorbate 80 (w/w).

37. The method of paragraph 36, wherein the MASP-3 inhibitory antibody, or antigen-binding fragment thereof, is included in the pharmaceutical composition at a concentration of 110 mg/mL±5%.

38. The method of any one of paragraphs 1-37, wherein the subject receives both a MASP-3 inhibitory agent and a second complement inhibitor.

39. The method of paragraph 38, wherein the second complement inhibitor is a C5 inhibitor.

40. The method of paragraph 39, wherein the C5 inhibitor is eculizumab, ravulizumab, or a biosimilar of either eculizumab or ravulizumab.

41. A method for treating a human subject suffering from complement 3 glomerulopathy (C3G) or idiopathic immune complex-mediated glomerulonephritis (ICGN), the method comprising administering to the subject an amount of a MASP-3 inhibitory agent sufficient to inhibit alternative pathway complement activation.

42. The method of paragraph 41, wherein the MASP-3 inhibitory agent is an anti-MASP-3 antibody or antigen-binding fragment thereof.

43. The method of paragraph 42, wherein the anti-MASP-3 antibody or antigen-binding fragment thereof comprises a heavy chain variable region comprising an HCDR1 having the sequence set forth as SEQ ID NO:3, an HCDR2 having the sequence set forth as SEQ ID NO:4 or 11, and an HCDR3 having the sequence set forth as SEQ ID NO:5 and a light chain variable region comprising an LCDR1 having the sequence set forth as SEQ ID NO: 6 or 14, an LCDR2 having the sequence set forth as SEQ ID NO:7, and an LCDR3 having the sequence set forth as SEQ ID NO:8.

44. The method of paragraph 41, wherein the anti-MASP-3 antibody or antigen-binding fragment thereof comprises an HCDR1 having the sequence set forth as SEQ ID NO:3, an HCDR2 having the sequence set forth as SEQ ID NO:4, an HCDR3 having the sequence set forth as SEQ ID NO:5, an LCDR1 having the sequence set forth as SEQ ID NO:6, an LCDR2 having the sequence set forth as SEQ ID NO:7, and an LCDR3 having the sequence set forth as SEQ ID NO:8.

45. The method of paragraph 44, wherein the anti-MASP-3 antibody or antigen-binding fragment thereof comprises a VH having the sequence set forth as SEQ ID NO:1 and a VL having the sequence set forth as SEQ ID NO:2.

46. The method of paragraph 44, wherein the anti-MASP-3 antibody or antigen-binding fragment thereof comprises a VH having the sequence set forth as SEQ ID NO:12 and a VL having the sequence set forth as SEQ ID NO:10.

47. The method of paragraph 41, wherein the anti-MASP-3 antibody or antigen-binding fragment thereof comprises an HCDR1 having the sequence set forth as SEQ ID NO:3, an HCDR2 having the sequence set forth as SEQ ID NO: 11, an HCDR3 having the sequence set forth as SEQ ID NO:5, an LCDR1 having the sequence set forth as SEQ ID NO:6, an LCDR2 having the sequence set forth as SEQ ID NO:7, and an LCDR3 having the sequence set forth as SEQ ID NO:8.

48. The method of paragraph 47, wherein the anti-MASP-3 antibody or antigen-binding fragment thereof comprises a VH having the sequence set forth as SEQ ID NO:9 and a VL having the sequence set forth as SEQ ID NO:10.

49. The method of paragraph 41, wherein the anti-MASP-3 antibody or antigen-binding fragment thereof comprises an HCDR1 having the sequence set forth as SEQ ID NO:3, an HCDR2 having the sequence set forth as SEQ ID NO:11, and an HCDR3 having the sequence set forth as SEQ ID NO:5, an LCDR1 having the sequence set forth as SEQ ID NO:14, an LCDR2 having the sequence set forth as SEQ ID NO:7, and an LCDR3 having the sequence set forth as SEQ ID NO:8.

50. The method of paragraph 49, wherein the anti-MASP-3 antibody or antigen-binding fragment thereof comprises a VH having the sequence set forth as SEQ ID NO:9 and a VL having the sequence set forth as SEQ ID NO:13.

51. The method of paragraph 41, wherein the anti-MASP-3 antibody or antigen-binding fragment thereof comprises an HCDR1 having the sequence set forth as SEQ ID NO:3, an HCDR2 having the sequence set forth as SEQ ID NO:4, an HCDR3 having the sequence set forth as SEQ ID NO:5, an LCDR1 having the sequence set forth as SEQ ID NO:14, an LCDR2 having the sequence set forth as SEQ ID NO:7, and an LCDR3 having the sequence set forth as SEQ ID NO:8.

52. The method of paragraph 51, wherein the anti-MASP-3 antibody or antigen-binding fragment thereof comprises a VH having the sequence set forth as SEQ ID NO:12 and a VL having the sequence set forth as SEQ ID NO:13.

53. The method of paragraph 52, wherein the anti-MASP-3 antibody or antigen-binding fragment thereof comprises a light chain having the sequence set forth as SEQ ID NO:15 and a heavy chain having the sequence set forth as SEQ ID NO:16

54. The method of any one of paragraphs 41-53, wherein the MASP-3 inhibitory agent is administered subcutaneously or intravenously.

55. The method of any one of paragraphs 41-54, wherein the MASP-3 inhibitory agent is administered at intervals of 4 to 16 weeks.

56. The method of paragraph 55, wherein the MASP-3 inhibitory agent is administered at intervals of 6 to 12 weeks.

57. The method of paragraph 55, wherein the MASP-3 inhibitory agent is administered at intervals of 4 weeks.

58. The method of paragraph 55, wherein the MASP-3 inhibitory agent is administered at intervals of 8 weeks.

59. The method of paragraph 55, wherein the MASP-3 inhibitory agent is administered at intervals of 12 weeks.

60. The method of any one of paragraphs 41-59, wherein the MASP-3 inhibitory agent is administered at a dosage of 0.1 mg/kg to 50 mg/kg.

61. The method of paragraph 60, wherein the MASP-3 inhibitory agent is administered at a dosage of 1 mg/kg to 25 mg/kg.

62. The method of paragraph 61, wherein the MASP-3 inhibitory agent is administered at a dosage of 1.0 mg/kg to 15.0 mg/kg.

63. The method of paragraph 61, wherein the MASP-3 inhibitory agent is administered at a dosage of about 1.0 mg/kg.

64. The method of paragraph 61, wherein the MASP-3 inhibitory agent is administered at a dosage of about 3.0 mg/kg.

65. The method of paragraph 61, wherein the MASP-3 inhibitory agent is administered at a dosage of about 5.0 mg/kg.

66. The method of paragraph 61, wherein the MASP-3 inhibitory agent is administered at a dosage of about 7.0 mg/kg.

67. The method of paragraph 61, wherein the MASP-3 inhibitory agent is administered at a dosage of about 10 mg/kg.

68. The method of paragraph 61, wherein the MASP-3 inhibitory agent is administered at a dosage of about 12 mg/kg.

69. The method of paragraph 61, wherein the MASP-3 inhibitory agent is administered at a dosage of about 15 mg/kg.

70. The method of paragraph 61, wherein the MASP-3 inhibitory agent is administered at a dosage of about 17 mg/kg.

71. The method of paragraph 61, wherein the MASP-3 inhibitory agent is administered at a dosage of about 20 mg/kg.

72. The method of any one of paragraphs 41-71, wherein a pharmaceutical composition is administered to the subject, the pharmaceutical composition comprising a MASP-3 inhibitory antibody, or antigen-binding fragment thereof, in an aqueous solution.

73. The method of paragraph 72, wherein the pharmaceutical composition comprises a MASP-3 inhibitory antibody, or antigen-binding fragment thereof, in an aqueous solution comprising a buffer system having a pH of 6.0±5%, 20±5% mM histidine, 100±5% mg/mL sucrose, and 0.035±5% polysorbate 80 (w/w).

74. The method of paragraph 73, wherein the MASP-3 inhibitory antibody, or antigen-binding fragment thereof, is included in the pharmaceutical composition at a concentration of 110 mg/mL±5%.

75. The use of a MASP-3 inhibitory agent in treatment of PNH, C3G, or idiopathic ICGN, wherein the MASP-3 inhibitory agent is an anti-MASP-3 antibody or antigen-binding fragment thereof.

76. The use of paragraph 75, wherein the anti-MASP-3 antibody or antigen-binding fragment thereof comprises a heavy chain variable region comprising an HCDR1 having the sequence set forth as SEQ ID NO:3, an HCDR2 having the sequence set forth as SEQ ID NO:4 or 11, and an HCDR3 having the sequence set forth as SEQ ID NO:5 and a light chain variable region comprising an LCDR1 having the sequence set forth as SEQ ID NO: 6 or 14, an LCDR2 having the sequence set forth as SEQ ID NO:7, and an LCDR3 having the sequence set forth as SEQ ID NO:8.

77. The use of a MASP-3 inhibitory agent in the manufacture of a medicament for treating PNH, C3G, or idiopathic ICGN, wherein the MASP-3 inhibitory agent is an anti-MASP-3 antibody or antigen-binding fragment thereof.

78. The use of paragraph 77, wherein the anti-MASP-3 antibody or antigen-binding fragment thereof comprises a heavy chain variable region comprising an HCDR1 having the sequence set forth as SEQ ID NO:3, an HCDR2 having the sequence set forth as SEQ ID NO:4 or 11, and an HCDR3 having the sequence set forth as SEQ ID NO:5 and a light chain variable region comprising an LCDR1 having the sequence set forth as SEQ ID NO: 6 or 14, an LCDR2 having the sequence set forth as SEQ ID NO:7, and an LCDR3 having the sequence set forth as SEQ ID NO:8.

1. A method for treating a human subject suffering from paroxysmal nocturnal hemoglobinuria (PNH), the method comprising administering to the subject an amount of a MASP-3 inhibitory agent sufficient to inhibit alternative pathway complement activation.

2. The method of claim 1, wherein the subject exhibits a sub-optimal response to treatment with a C5 inhibitor.

3. The method of claim 2, wherein the C5 inhibitor is eculizumab, ravulizumab, or a biosimilar to either eculizumab or ravulizumab.

4. The method of claim 2, wherein the subject exhibits a hemoglobin level of less than 10.5 g/dL in response to C5 inhibitor treatment.

5. The method of claim 1, wherein the MASP-3 inhibitory agent is an anti-MASP-3 antibody or antigen-binding fragment thereof.

6. The method of claim 5, wherein the anti-MASP-3 antibody or antigen-binding fragment thereof comprises a heavy chain variable region comprising an HCDR1 having the sequence set forth as SEQ ID NO:3, an HCDR2 having the sequence set forth as SEQ ID NO:4 or 11, and an HCDR3 having the sequence set forth as SEQ ID NO:5 and a light chain variable region comprising an LCDR1 having the sequence set forth as SEQ ID NO: 6 or 14, an LCDR2 having the sequence set forth as SEQ ID NO:7, and an LCDR3 having the sequence set forth as SEQ ID NO:8.

7. The method of claim 6, wherein the anti-MASP-3 antibody or antigen-binding fragment thereof comprises an HCDR1 having the sequence set forth as SEQ ID NO:3, an HCDR2 having the sequence set forth as SEQ ID NO:4, an HCDR3 having the sequence set forth as SEQ ID NO:5, an LCDR1 having the sequence set forth as SEQ ID NO:6, an LCDR2 having the sequence set forth as SEQ ID NO:7, and an LCDR3 having the sequence set forth as SEQ ID NO:8.

8. (canceled)

9. (canceled)

10. The method of claim 6, wherein the anti-MASP-3 antibody or antigen-binding fragment thereof comprises an HCDR1 having the sequence set forth as SEQ ID NO:3, an HCDR2 having the sequence set forth as SEQ ID NO: 11, an HCDR3 having the sequence set forth as SEQ ID NO:5, an LCDR1 having the sequence set forth as SEQ ID NO:6, an LCDR2 having the sequence set forth as SEQ ID NO:7, and an LCDR3 having the sequence set forth as SEQ ID NO:8.

11. (canceled)

12. The method of claim 6, wherein the anti-MASP-3 antibody or antigen-binding fragment thereof comprises an HCDR1 having the sequence set forth as SEQ ID NO:3, an HCDR2 having the sequence set forth as SEQ ID NO:11, and an HCDR3 having the sequence set forth as SEQ ID NO:5, an LCDR1 having the sequence set forth as SEQ ID NO:14, an LCDR2 having the sequence set forth as SEQ ID NO:7, and an LCDR3 having the sequence set forth as SEQ ID NO:8.

13. (canceled)

14. The method of claim 6, wherein the anti-MASP-3 antibody or antigen-binding fragment thereof comprises an HCDR1 having the sequence set forth as SEQ ID NO:3, an HCDR2 having the sequence set forth as SEQ ID NO:4, an HCDR3 having the sequence set forth as SEQ ID NO:5, an LCDR1 having the sequence set forth as SEQ ID NO:14, an LCDR2 having the sequence set forth as SEQ ID NO:7, and an LCDR3 having the sequence set forth as SEQ ID NO:8.

15. (canceled)

16. (canceled)

17. The method of claim 1, wherein the MASP-3 inhibitory agent is administered subcutaneously or intravenously.

18. The method of claim 1, wherein the MASP-3 inhibitory agent is administered at intervals of 4 to 16 weeks.

19. (canceled)

20. (canceled)

21. (canceled)

22. The method of claim 1, wherein the MASP-3 inhibitory agent is administered at a dosage of 1 mg/kg to 25 mg/kg.

23. The method of claim 22, wherein the MASP-3 inhibitory agent is administered at a dosage of 1 mg/kg to 15.0 mg/kg.

24. (canceled)

25. (canceled)

26. (canceled)

27. (canceled)

28. The method of claim 1, wherein the subject receives both a MASP-3 inhibitory agent and a second complement inhibitor.

29. The method of claim 28, wherein the second complement inhibitor is a C5 inhibitor.

30. (canceled)

31. A method for treating a human subject suffering from complement 3 glomerulopathy (C3G) or idiopathic immune complex-mediated glomerulonephritis (ICGN), the method comprising administering to the subject an amount of a MASP-3 inhibitory agent sufficient to inhibit alternative pathway complement activation.

32. The method of claim 31, wherein the MASP-3 inhibitory agent is an anti-MASP-3 antibody or antigen-binding fragment thereof.

33. The method of claim 32, wherein the anti-MASP-3 antibody or antigen-binding fragment thereof comprises a heavy chain variable region comprising an HCDR1 having the sequence set forth as SEQ ID NO:3, an HCDR2 having the sequence set forth as SEQ ID NO:4 or 11, and an HCDR3 having the sequence set forth as SEQ ID NO:5 and a light chain variable region comprising an LCDR1 having the sequence set forth as SEQ ID NO: 6 or 14, an LCDR2 having the sequence set forth as SEQ ID NO:7, and an LCDR3 having the sequence set forth as SEQ ID NO:8.

34. The method of claim 33, wherein the anti-MASP-3 antibody or antigen-binding fragment thereof comprises an HCDR1 having the sequence set forth as SEQ ID NO:3, an HCDR2 having the sequence set forth as SEQ ID NO:4, an HCDR3 having the sequence set forth as SEQ ID NO:5, an LCDR1 having the sequence set forth as SEQ ID NO:6, an LCDR2 having the sequence set forth as SEQ ID NO:7, and an LCDR3 having the sequence set forth as SEQ ID NO:8.

35. (canceled)

36. (canceled)

37. The method of claim 33, wherein the anti-MASP-3 antibody or antigen-binding fragment thereof comprises an HCDR1 having the sequence set forth as SEQ ID NO:3, an HCDR2 having the sequence set forth as SEQ ID NO: 11, an HCDR3 having the sequence set forth as SEQ ID NO:5, an LCDR1 having the sequence set forth as SEQ ID NO:6, an LCDR2 having the sequence set forth as SEQ ID NO:7, and an LCDR3 having the sequence set forth as SEQ ID NO:8.

38. (canceled)

39. The method of claim 33, wherein the anti-MASP-3 antibody or antigen-binding fragment thereof comprises an HCDR1 having the sequence set forth as SEQ ID NO:3, an HCDR2 having the sequence set forth as SEQ ID NO:11, and an HCDR3 having the sequence set forth as SEQ ID NO:5, an LCDR1 having the sequence set forth as SEQ ID NO:14, an LCDR2 having the sequence set forth as SEQ ID NO:7, and an LCDR3 having the sequence set forth as SEQ ID NO:8.

40. (canceled)

41. The method of claim 33, wherein the anti-MASP-3 antibody or antigen-binding fragment thereof comprises an HCDR1 having the sequence set forth as SEQ ID NO:3, an HCDR2 having the sequence set forth as SEQ ID NO:4, an HCDR3 having the sequence set forth as SEQ ID NO:5, an LCDR1 having the sequence set forth as SEQ ID NO:14, an LCDR2 having the sequence set forth as SEQ ID NO:7, and an LCDR3 having the sequence set forth as SEQ ID NO:8.

42. (canceled)

43. (canceled)

44. The method of claim 31, wherein the MASP-3 inhibitory agent is administered subcutaneously or intravenously.

45. The method of claim 31, wherein the MASP-3 inhibitory agent is administered at intervals of 4 to 16 weeks.

46. (canceled)

47. (canceled)

48. (canceled)

49. (canceled)

50. The method of claim 31, wherein the MASP-3 inhibitory agent is administered at a dosage of 1 mg/kg to 25.0 mg/kg.

51. The method of claim 50, wherein the MASP-3 inhibitory agent is administered at a dosage of 1.0 mg/kg to 15.0 mg/kg.

52. (canceled)

53. (canceled)

54. (canceled)

55. (canceled)

56. (canceled)

57. The method of claim 1 or claim 31, wherein a pharmaceutical composition is administered to the subject, the pharmaceutical composition comprising a MASP-3 inhibitory antibody, or antigen-binding fragment thereof, in an aqueous solution.

58. The method of claim 57, wherein the pharmaceutical composition comprises a MASP-3 inhibitory antibody, or antigen-binding fragment thereof, in an aqueous solution comprising a buffer system having a pH of 6.0±5%, 20±5% mM histidine, 100±5% mg/mL sucrose, and 0.035±5% polysorbate 80 (w/w).

59. The method of claim 57, wherein the MASP-3 inhibitory antibody, or antigen-binding fragment thereof, is included in the pharmaceutical composition at a concentration of 110 mg/mL±5%.

60. (canceled)

61. (canceled)

62. (canceled)

63. (canceled)

64. (canceled)

65. (canceled)
